(19) United States
(12) Patent Application Publication
(10) Pub. No.: US 2007/0043390 A1
(43) Pub. Date: Feb. 22, 2007

(54) DELIVERY CATHETER

(75) Inventor: John Neilan, Gort (IE)

Correspondence Address: SUGHRUE MION, PLLC, 2100 PENNSYLVANIA AVENUE, N.W., SUITE 800, WASHINGTON, DC, 20037, US

(73) Assignee: Salviac Limited

(21) Appl. No.: 11/465,615

(22) Filed: Aug. 18, 2006

Related U.S. Application Data
(60) Provisional application No. 60/709,155, filed on Aug. 18, 2005.

(51) Int. Cl. A61M 29/00

(52) U.S. Cl. 606/200

(57) ABSTRACT

A delivery catheter, for delivering an embolic protection filter through a vasculature over a guidewire, comprises a distal pod (1) which acts as a restraining sheath to restrain the embolic protection filter in a collapsed configuration during delivery. The pod (1) has a proximal portion (2), a distal portion (3) and a flared portion (4) intermediate the proximal portion (2) and the distal portion (3). The pod (1) comprises a layer (39) of an intractable material, such as polyamide, and a low coefficient of friction material layer (40), such as of polytetrafluoroethylene. Each layer (39, 40) is integrally formed and extends from the proximal portion (2) to the distal portion (3). A marker band (5) is mounted to the exterior surface of the proximal portion (2) to aid in visualisation of the pod (1).

This application claims benefit under 35 U.S.C. § 119(e) of Provisional Application No. 60/709,155 filed Aug. 18, 2005, the entire disclosure of which is incorporated herein by reference.

### INTRODUCTION

This invention relates to a delivery catheter suitable for delivering an embolic protection filter to a desired site in a vasculature, and for deploying the embolic protection filter at the desired site. In one embodiment this invention relates to a delivery catheter, which is configured to facilitate rapid exchange of the catheter over a guidewire during delivery and deployment of an embolic protection filter.

Exchange of a catheter over a guidewire using a rapid exchange arrangement may enable an interventional procedure to be performed by a single operator in a fast, efficient manner.

This invention is aimed at providing an improved delivery catheter which is suitable for delivery and deployment of an embolic protection filter.

### STATEMENTS OF INVENTION

According to the invention there is provided a delivery catheter comprising a flared pod, the pod defining a reception space for an embolic protection filter, the pod having a proximal portion and a distal portion, the pod comprising at least one integrally formed layer extending from the proximal portion to the distal portion.

In one embodiment of the invention the pod is flared intermediate the proximal portion and the distal portion. Along the proximal portion, the pod may have a substantially constant diameter. Along the distal portion, the pod may have a substantially constant diameter.

In one case the outer diameter of the distal portion of the pod is greater than the outer diameter of the proximal portion of the pod. The inner diameter of the distal portion of the pod may be greater than the inner diameter of the proximal portion of the pod.

In another case the pod is necked. The wall thickness of the pod may vary along the pod. The wall thickness of the pod may decrease distally along the pod. The wall thickness of the pod may vary intermediate the proximal portion and the distal portion. The wall thickness of the distal portion of the pod may be approximately 40% of the wall thickness of the proximal portion of the pod. Along the proximal portion, the pod may have a substantially constant wall thickness. The wall thickness of the proximal portion of the pod may be in the range of from 0.00225″ to 0.00325″. The wall thickness of the proximal portion of the pod may be approximately 0.00275″. Along the distal portion, the pod may have a substantially constant wall thickness. The wall thickness of the distal portion of the pod may be in the range of from 0.0008″ to 0.0012″. The wall thickness of the distal portion of the pod may be approximately 0.0011″. The wall thickness of the proximal portion of the pod may be greater than the wall thickness of the distal portion of the pod.

In another embodiment the catheter comprises a visualisation element to aid visualisation of the catheter. The visualisation element may be located exterior of the pod. The visualisation element may be located along at least part of an exterior surface of the pod. The visualisation element may be mounted to the exterior surface of the pod. The visualisation element may be mounted to the proximal portion of the pod. The visualisation element may be mounted to an intermediate portion of the pod. The exterior surface of the pod may define a mounting region for receiving at least part of the visualisation element. By mounting the visualisation element in the mounting region on the exterior surface of the pod, the overall crossing profile of the catheter may be minimised. The exterior surface of the visualisation element received in the mounting region may be substantially flush with the exterior surface of the pod adjacent to the mounting region. The mounting region may comprise a recess. The visualisation element may be bonded to the exterior surface of the pod.

In one case the visualisation element comprises a marker band. The visualisation element may be at least partially of a radiopaque material.

In one embodiment the pod comprises a first layer and a second layer. Each layer may be integrally formed. At the proximal portion the pod may comprise the first layer and the second layer. At the distal portion the pod may comprise the first layer and the second layer. Each layer may extend from the proximal portion to the distal portion.

In one case the first layer is located radially outwardly of the second layer.

In another case the wall thickness of the first layer is greater than the wall thickness of the second layer. The wall thickness of the first layer may be approximately 10 times greater than the wall thickness of the second layer. Along the proximal portion of the pod, the wall thickness of the first layer may be in the range of from 0.00225″ to 0.00275″. Along the proximal portion of the pod, the wall thickness of the first layer may be approximately 0.0025″. Along the proximal portion of the pod, the wall thickness of the second layer may be approximately 0.00025″. Along the distal portion of the pod, the wall thickness of the first layer may be approximately 0.001″. Along the distal portion of the pod, the wall thickness of the second layer may be approximately 0.0001″.

In one case the first layer is a strengthening layer. The first layer may be at least partially of an intractable material. The first layer may be at least partially of a non-thermoplastic material. The first layer may be at least partially of a thermoset material. The first layer may be at least partially of polyamide.

In another case the second layer is at least partially of a low coefficient of friction material. The low-friction layer of the pod assists in delivery of an embolic protection filter from within the reception space. The second layer may be at least partially of polytetrafluoroethylene.

In a further embodiment the catheter comprises a catheter shaft, the pod being movable relative to the catheter shaft to facilitate deployment of an embolic protection filter from within the reception space. The catheter may comprise an operating element to facilitate movement of the pod relative to the catheter shaft. The operating element enables a user to achieve a steady, accurate deployment at a desired site in a vasculature while ensuring the overall crossing profile of the delivery catheter is kept to a minimum. The operating element may be coupled to the pod. The operating element may be coupled to the proximal portion of the pod.

In addition during advancement of the delivery catheter through a vasculature, the control wire may bend around its own neutral axis. This results in the contribution of the control wire to the overall stiffness of the catheter being kept to a minimum to obtain a highly trackable delivery catheter.

In one case the catheter comprises an engagement element for engaging an embolic protection filter in the reception space upon movement of the pod relative to the catheter shaft. The engagement element may be attached to the catheter shaft. The engagement element may be bonded to the catheter shaft. The catheter may comprise a constraint around the region of attachment of the engagement element to the catheter shaft. The constraint assists in maintaining the overall profile at the attachment region constant and assists in minimising the overall profile at the attachment region. The constraint may comprise a sleeve.

In another case the catheter has a proximal guidewire opening located a substantial distance distally of a proximal end of the catheter for rapid exchange of the catheter over a guidewire.

In another aspect the invention provides a delivery catheter comprising:—
a pod, the pod defining a reception space for an embolic protection filter; and
a visualisation element to aid visualisation of the catheter;
the visualisation element being located exterior of the pod.

In another aspect of the invention there is provided a delivery catheter comprising a pod, the pod defining a reception space for an embolic protection filter, the pod comprising a first layer, the first layer being at least partially of an intractable material.

The intractable material enables the first layer to be manufactured with particularly accurate tolerances. This enables the pod to be manufactured with a particularly small diameter.

In one embodiment of the invention the first layer is at least partially of a non-thermoplastic material. The first layer may be at least partially of a thermoset material. The first layer may be at least partially of polyamide. The pod may comprise a second layer. The second layer may be at least partially of a low coefficient of friction material. The second layer may be at least partially of polytetrafluoroethylene.

The invention also provides in a further aspect a delivery catheter comprising a pod, the pod defining a reception space for an embolic protection filter, the pod comprising a first layer and a second layer at a distal portion of the pod.

According to another aspect of the invention, there is provided a delivery catheter comprising:—
a pod, the pod defining a reception space for an embolic protection filter;
a catheter shaft, the pod being movable relative to the catheter shaft to facilitate deployment of an embolic protection filter from within the reception space;
an engagement element for engaging an embolic protection filter in the reception space upon movement of the pod relative to the catheter shaft;
the engagement element being attached to the catheter shaft; and
a constraint around the region of attachment of the engagement element to the catheter shaft.

In a further aspect of the invention there is provided a delivery catheter comprising:—
a pod, the pod defining a reception space for an embolic protection filter; and
a stop to limit movement of an embolic protection filter proximally relative to the pod.

In one embodiment the stop is configured to limit movement of an embolic protection filter proximally relative to the pod upon loading of the embolic protection filter into the pod.

The stop may comprise a marker band. The stop may comprise a visualisation element. The stop may be located exterior of the pod.

The invention provides in a further aspect a method of producing a delivery catheter, the method comprising the steps of:—
providing a delivery catheter pod comprising at least one integrally formed layer extending from a proximal portion of the pod to a distal portion of the pod; and
flaring at least part of the pod.

In one embodiment the pod is flared by inserting a flaring mandrel into the pod. The method may comprise the step of heating the pod during flaring.

In one case the method comprises the step of necking at least part of the pod. The pod may be at least partially necked after flaring. The pod may be at least partially necked before flaring. The pod may be necked by engaging the pod using a necking clamp. The method may comprise the step of locating emery paper between the pod and the necking clamp before necking. A mandrel may be inserted into the pod before necking. The method may comprise the step of heating the pod during necking.

In another embodiment the method comprises the step of annealing the pod after necking.

In one case the method comprises the step of forming the pod from a first layer. The first layer may be at least partially of an intractable material. The method may comprises the step of forming the pod from the first layer and a second layer. The pod may be formed by dipping the second layer in solid form into a quantity of the first layer in liquid form. The second layer in solid form may be formed by extrusion.

In a further embodiment the method comprises the step of locating a visualisation element exterior of the pod. The visualisation element may be located along an exterior surface of the pod. The visualisation element may be mounted to the exterior surface of the pod. The visualisation element may be slid along the exterior surface of the pod to a desired mounting position. The visualisation element may be slid distally along the exterior surface of the pod from a proximal end of the pod. The visualisation element may be slid proximally along the exterior surface of the pod from a distal end of the pod. The visualisation element may be bonded to the exterior surface of the pod. The pod may be flared before locating the visualisation element. The pod may be flared after locating the visualisation element.

In another embodiment the method comprises the step of attaching an engagement element to a catheter shaft. The engagement element may be bonded to the catheter shaft. The method may comprise the step of locating a constraint around the region of attachment of the engagement element to the catheter shaft. The constraint may be located around the region of attachment before attaching the engagement element to the catheter shaft.

In a further aspect, the invention provides a method of producing a delivery catheter, the method comprising the steps of:—
providing a delivery catheter pod; and
locating a visualisation element exterior of the pod.

The invention provides in a further aspect a method of providing a delivery catheter, the method comprising the step of:—
forming a delivery catheter pod from a first layer;
the first layer being at least partially of an intractable material.

The delivery catheter of the invention is particularly suitable for delivering an embolic protection filter through a vasculature over a guidewire, and for deploying the filter at a desired site in the vasculature.

### BRIEF DESCRIPTION OF THE DRAWINGS

The invention will be more clearly understood from the following description of some embodiments thereof, given by way of example only, with reference to the accompanying drawings, in which:—

FIG. 1(a) is a side view of a delivery catheter according to the invention;

FIG. 1(b) is a partially cut-away, perspective view of a part of the catheter of FIG. 1(a);

FIG. 1(c) is a side view of a pod and a visualisation element of the catheter of FIG. 1(a);

FIG. 2 is a view along line A-A in FIG. 1(c);

FIG. 3 is an enlarged view of detail B in FIG. 2;

FIGS. 3(a) to 3(c) are cross-sectional, side views illustrating production of the pod of FIG. 1(c);

FIG. 3(d) is an enlarged view of detail A in FIG. 3(c);

FIG. 3(e) is an enlarged view of detail B in FIG. 3(c);

FIG. 3(i) is a side view of the catheter of FIG. 1(b), in use;

FIGS. 3(j) to 3(l) are views similar to FIGS. 3(c) to 3(e) of a pod and a visualisation element of another delivery catheter according to the invention;

FIGS. 3(m) to 3(o) are views similar to FIGS. 3(c) to 3(e) of a pod and a visualisation element of a further delivery catheter according to the invention;

FIG. 3(p) is a side view of another delivery catheter according to the invention;

FIG. 3(q) is a partially cut-away, perspective view of a part of the catheter of FIG. 3(p);

FIGS. 4 to 6 are views similar to FIGS. 1(c) to 3 of a pod and a visualisation element of the catheter of FIG. 3(p);

FIGS. 6(a) to 6(d) and 6(g) are cross-sectional, side views illustrating production of the pod of FIG. 4;

FIG. 6(e) is an enlarged view of detail A in FIG. 6(d);

FIG. 6(f) is an enlarged view of detail B in FIG. 6(d);

FIG. 6(k) is a side view of the catheter of FIG. 3(q), in use;

FIG. 7 is a partially cross-sectional, side view of another delivery catheter according to the invention;

FIG. 8 is a cross-sectional, side view of the catheter of FIG. 7;

FIGS. 9 and 10 are views similar to FIGS. 7 and 8 of another delivery catheter according to the invention;

FIGS. 11 and 12 are views similar to FIGS. 7 and 8 of another delivery catheter according to the invention;

FIGS. 13 and 14 are views similar to FIGS. 7 and 8 of a filter delivery catheter according to the invention;

FIGS. 15 and 16 are views similar to FIGS. 7 and 8 of another delivery catheter according to the invention;

FIGS. 17 and 18 are views similar to FIGS. 7 and 8 of another delivery catheter according to the invention;

FIGS. 19 and 20 are views similar to FIGS. 7 and 8 of a further delivery catheter according to the invention;

FIGS. 21 and 22 are views similar to FIGS. 7 and 8 of another delivery catheter according to the invention;

FIG. 23 is a partially cross-sectional, side view of another delivery catheter according to the invention;

FIG. 24 is a view similar to FIG. 23 of another delivery catheter according to the invention;

FIG. 25 is a view similar to FIG. 23 of another delivery catheter according to the invention;

FIG. 26 is a cross-sectional, side view of another delivery catheter according to the invention;

FIG. 27 is a view similar to FIG. 26 of another delivery catheter according to the invention;

FIG. 28 is a view similar to FIG. 26 of another delivery catheter according to the invention;

FIG. 29 is a view similar to FIG. 26 of a further delivery catheter according to the invention;

FIG. 30 is a view similar to FIG. 26 of another delivery catheter according to the invention;

FIG. 31 is a view similar to FIG. 26 of another delivery catheter according to the invention;

FIG. 32 is a view similar to FIG. 26 of a further delivery catheter according to the invention;

FIG. 33 is a view similar to FIG. 26 of another delivery catheter according to the invention;

FIG. 34 is a view similar to FIG. 26 of another delivery catheter according to the invention;

FIG. 35 is a view similar to FIG. 26 of a further delivery catheter according to the invention;

FIG. 36 is a perspective view of a proximal end of another delivery catheter according to the invention;

FIG. 37 is a perspective view of the catheter of FIG. 36, in use;

FIGS. 38 and 39 are views similar to FIGS. 36 and 37 of another delivery catheter according to the invention;

FIGS. 40 and 41 are views similar to FIGS. 36 and 37 of another delivery catheter according to the invention;

FIGS. 42 and 43 are views similar to FIGS. 36 and 37 of another delivery catheter according to the invention; and

FIGS. 44 and 45 are views similar to FIGS. 36 and 37 of a further delivery catheter according to the invention.

### DETAILED DESCRIPTION

Referring to the drawings, there is illustrated a delivery catheter according to the invention which is suitable for delivery of an embolic protection filter through a vasculature over a guidewire, and for deployment of the filter at a desired site in the vasculature.

In some embodiments of the invention the delivery catheter is suitable for rapid exchange over a guidewire during delivery and deployment of an embolic protection filter in a vasculature, and during withdrawal of the delivery catheter after deployment. In these embodiments, the delivery catheter extends between a proximal end and a distal end, and the catheter defines an opening in a sidewall of the catheter, and an opening at the distal end of the catheter. A guidewire lumen extends between these openings to enable passage of a guidewire through the lumen, and thereby facilitate rapid exchange of the delivery catheter over the guidewire.

A distal portion of the catheter defines a reception space for an embolic protection filter during delivery of the filter through a vasculature, and at least one elongate actuator is provided extending along the catheter to facilitate deployment of the filter from within the reception space.

In some embodiments of the invention the delivery catheter is suitable for delivery and deployment of a filter, which is received within the reception space but is separate and independent of the delivery catheter, and which is separate and independent of the rapid exchange guidewire. Examples of this type of filter are the embolic protection filters described in International patent application number PCT/IE01/00053 and International patent application number PCT/IE2006/000034, the relevant contents of which are incorporated herein by reference.

In the region adjacent the guidewire opening in the sidewall of the catheter, the actuator has a small cross-sectional area relative to the overall cross-sectional area of the delivery catheter. By providing such a thin, elongate actuator, this ensures that the guidewire opening in the sidewall of the catheter, which serves as the rapid exchange port for a guidewire, will not be obstructed or occluded by manipulation of the actuator upon deployment of a filter from within the reception space.

The delivery catheter according to the invention is particularly suitable for delivery and deployment of an expandable embolic protection filter. In this case, the distal portion of the catheter is provided by a sheath which restrains the embolic protection filter in a low-profile, collapsed configuration within the reception space during delivery to a desired site in a vasculature. The sheath is preferably thin-walled to minimise the overall crossing profile of the delivery catheter, especially during delivery of the embolic protection filter.

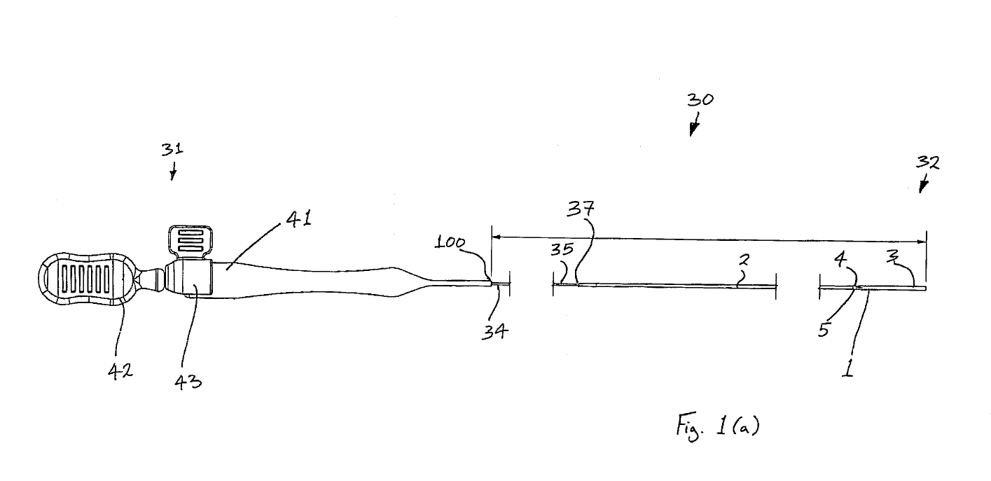

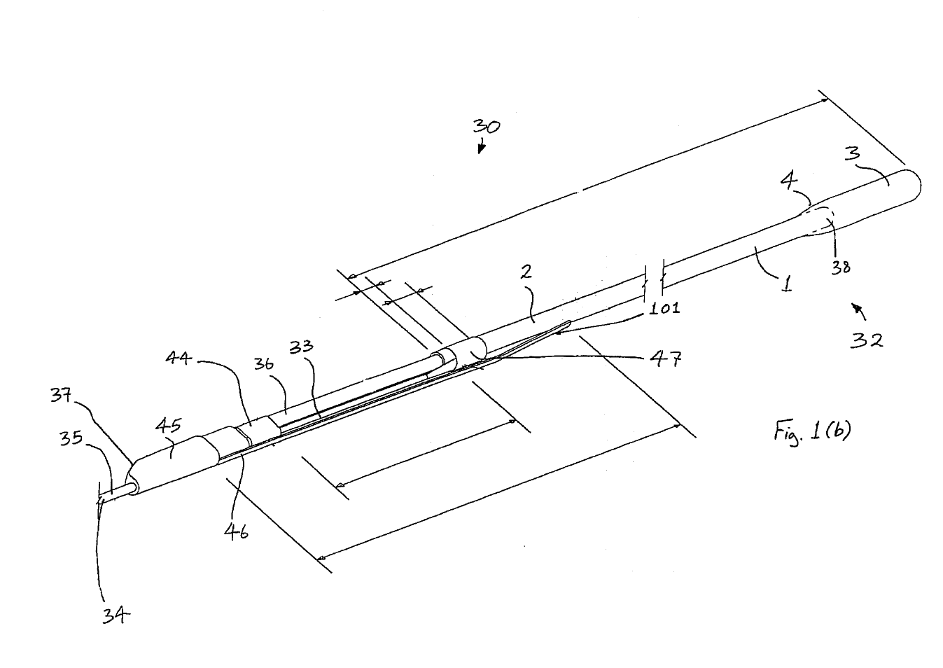

Referring to FIG. 1(a) to 3(i) there is illustrated a delivery catheter 30 according to the invention. The delivery catheter 30 comprises a catheter body 34 which extends between a proximal end 31 and a distal end 32, a distal pod 1 which acts as a restraining sheath at the distal end 32 of the catheter body 34, and an elongate actuator 33, which is provided in this case in the form of a stainless steel wire.

The catheter body 34 comprises a catheter shaft 35 in the form of a proximal hypotube portion, and an engagement element 36 in the form of a radially offset distal spring pusher. The pusher 36 is fixedly attached to the hypotube 35 in a side-by-side overlapping arrangement with the proximal end of the pusher 36 located proximally of the distal end of the hypotube 35. An overmould joiner 45 is provided in the region of overlap between the pusher 36 and the hypotube 35.

In this case the pusher 36 is bonded to the catheter shaft 35 by means of an adhesive. A constraining sleeve 44 is located around the region of attachment of the pusher 36 to the catheter shaft 35 before the pusher 36 is bonded to the catheter shaft 35. In this case the sleeve 44 is of the material polyethyleneterephthalate (PET).

The PET sleeve 44 covers the adhesive bond between the catheter shaft 35 and the pusher 36. This PET sleeve 44 is placed in position before the adhesive is applied to the catheter shaft 35 and to the pusher 36. The PET sleeve 44 keeps the profile of the bond constant and to a minimum. The PET sleeve 44 also helps to form an extended bonding surface from the RX port 37 when adhering the outer sleeve 46 to the RX port 37.

The pusher 36 has a guidewire lumen extending through the pusher 36 with an opening 37 at the proximal end of the lumen for passage of a guidewire through the lumen and out through the proximal guidewire opening 37. The opening 37 is provided in the wall of the overmould joiner 45. The delivery catheter 30 is thus configured to be passed over the guidewire in a rapid-exchange manner. The position of the RX port 37 is illustrated in FIGS. 1(a) and 1(b).

The pusher 36 may taper proximally inwardly at the opening 37 for a smooth crossing profile.

A protective sleeve 46 extends distally from the overmould joiner 45 over the wire 33 to protect the wire 33. The sleeve 46 is fixedly attached to the overmould joiner 45, for example by means of an adhesive bond. The length of the sleeve 46 from the distal end of the overmould joiner 45 to the distal end of the sleeve 40 may be approximately 75 mm. Along the distal portion 101 of the sleeve 46, the sleeve 46 tapers radially inwardly, as illustrated in FIG. 1(b). In particular, in this case along the distal 5 mm of the sleeve 46, the sleeve 46 tapers inwardly to an inner diameter of 0.0365″. In FIG. 1(b), the sleeve 46 is illustrated as being partially cut-away, for the purposes of clarity.

When assembled, the hypotube 35 and the pusher 36 are located substantially side-by-side. This side-by-side assembly of the hypotube 35 relative to the pusher 36 enables the guidewire to exit through the proximal guidewire opening 37 smoothly and substantially parallel to the longitudinal axis of the catheter 30. In particular the passage of the guidewire through the proximal guidewire opening 37 does not increase the overall profile of the catheter 30.

The pod 1 extends proximally over the pusher 36 towards the distal end of the hypotube 35.

The actuator wire 33 extends distally through an actuator lumen in the hypotube 35, out of the actuator lumen at the distal end of the hypotube 35, externally along the pusher 36 to the proximal end of the pod 1. The wire 33 is attached to the exterior surface of the pod 1, for example by bonding. A shrink-wrap polyimide tube 47 may be used to assist in attachment of the wire 33 to the pod 1. The wire 33 is adhesively bonded to the tube 47. The length of the tube 47 may be in the range of from 12 mm to 13 mm. The distance from the proximal end of the pod 1 to the proximal end of the tube 47 may be approximately 1 mm.

By attaching the wire 33 to the exterior of the pod 1, this arrangement provides for more space within the pusher lumen for guidewire passage.

In addition attachment of the actuator wire 33 to the exterior of the pod 1 is an easier step to achieve from a manufacturing viewpoint than attachment to the interior of the relatively long pod 1.

The pod 1 is movable in a sliding manner relative to the catheter body 34.

When the pod 1 extends distally of a distal end 38 of the spring pusher 36, the pod 1 defines an internal reception space. A collapsed embolic protection filter may be received within the reception space, where the filter will be restrained by the pod 1 in a low-profile configuration during delivery to a desired site in a vasculature.

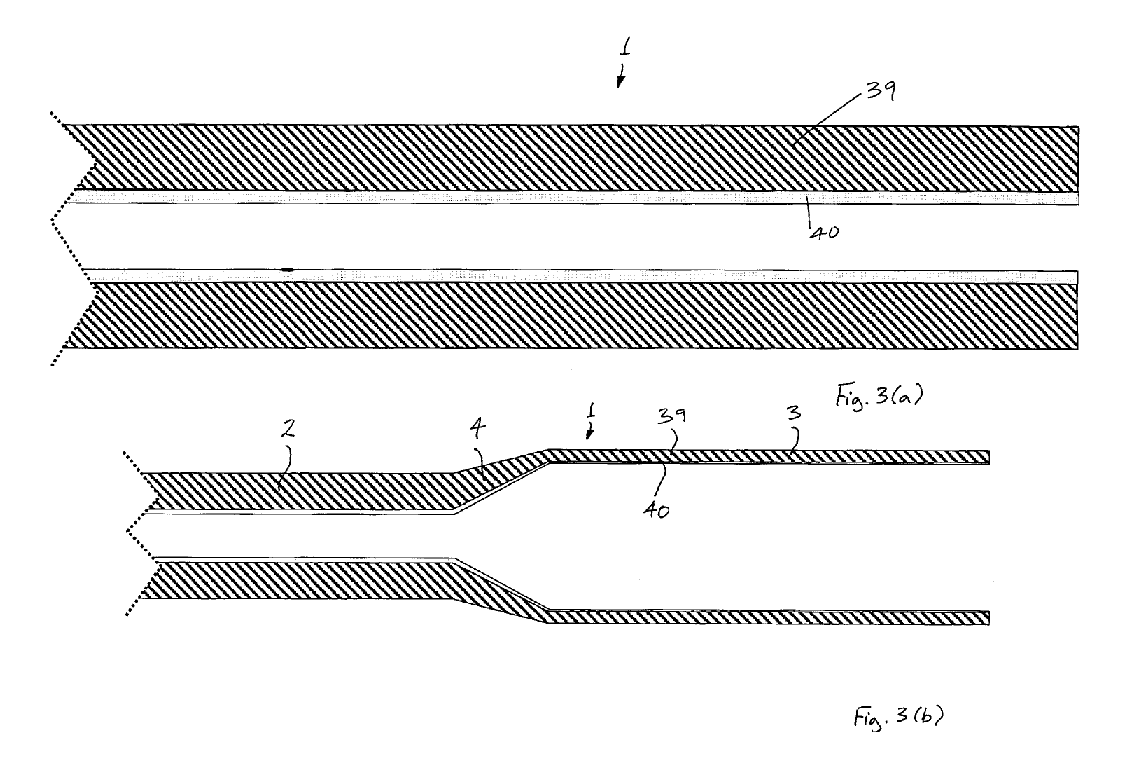

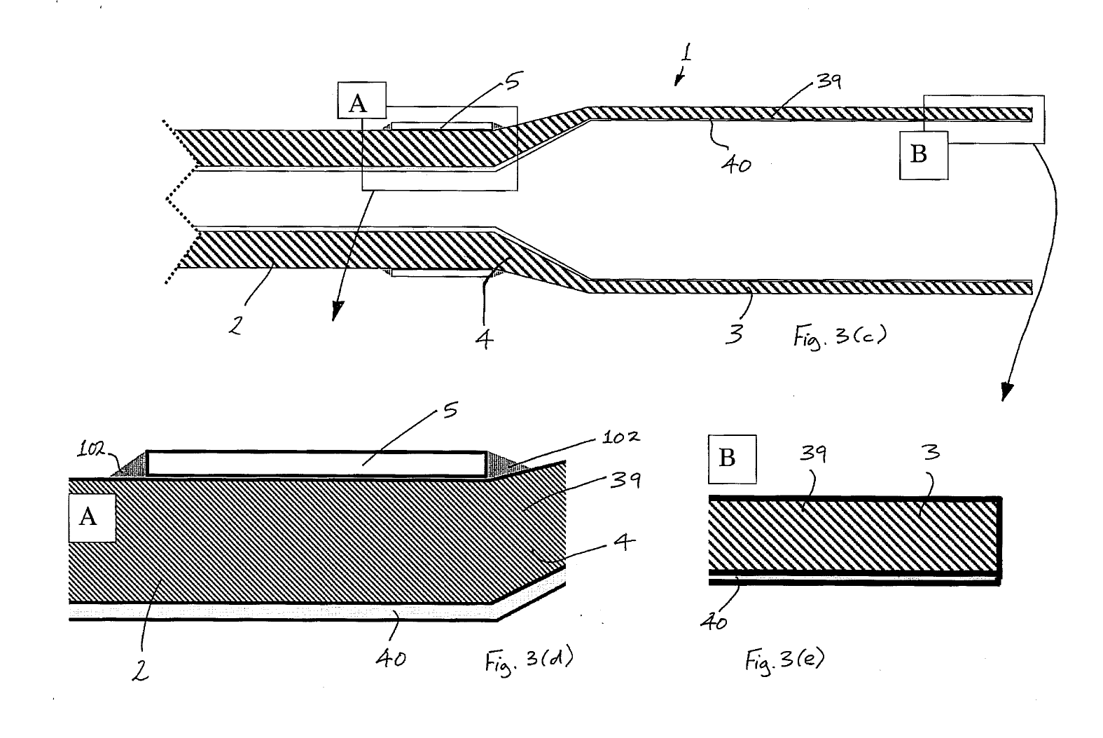

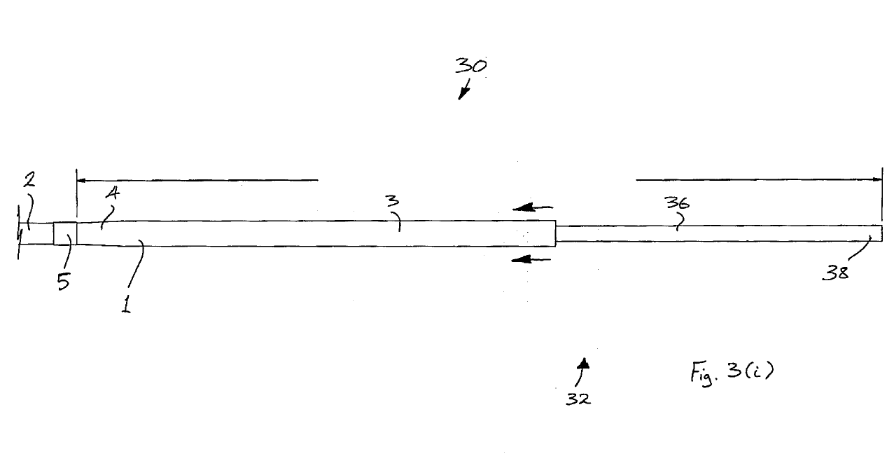

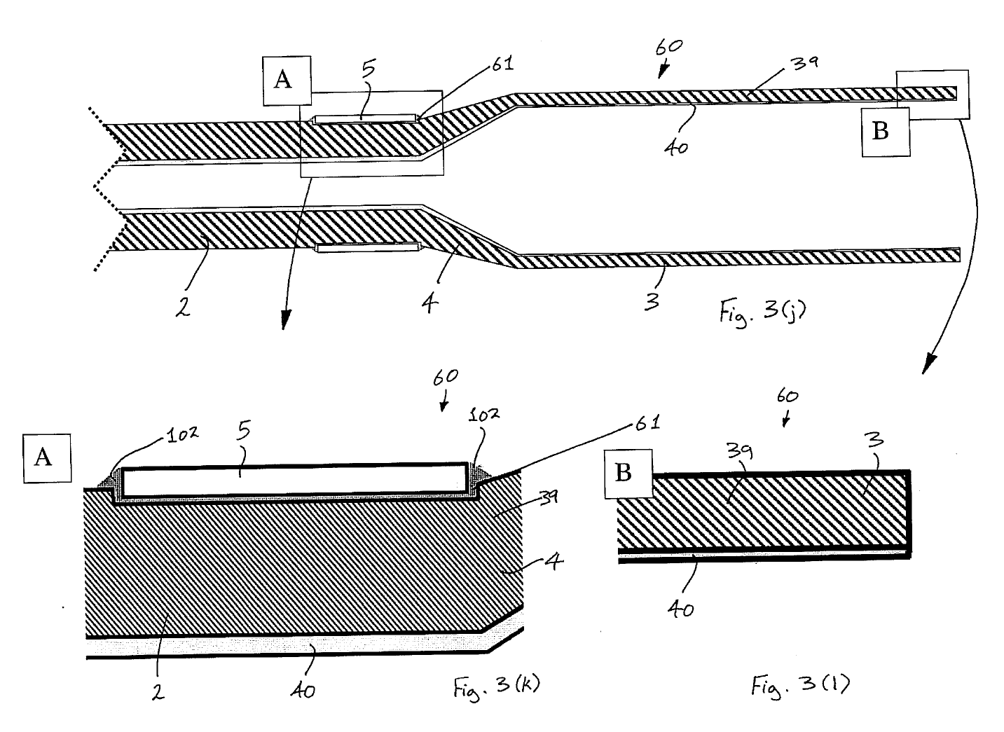

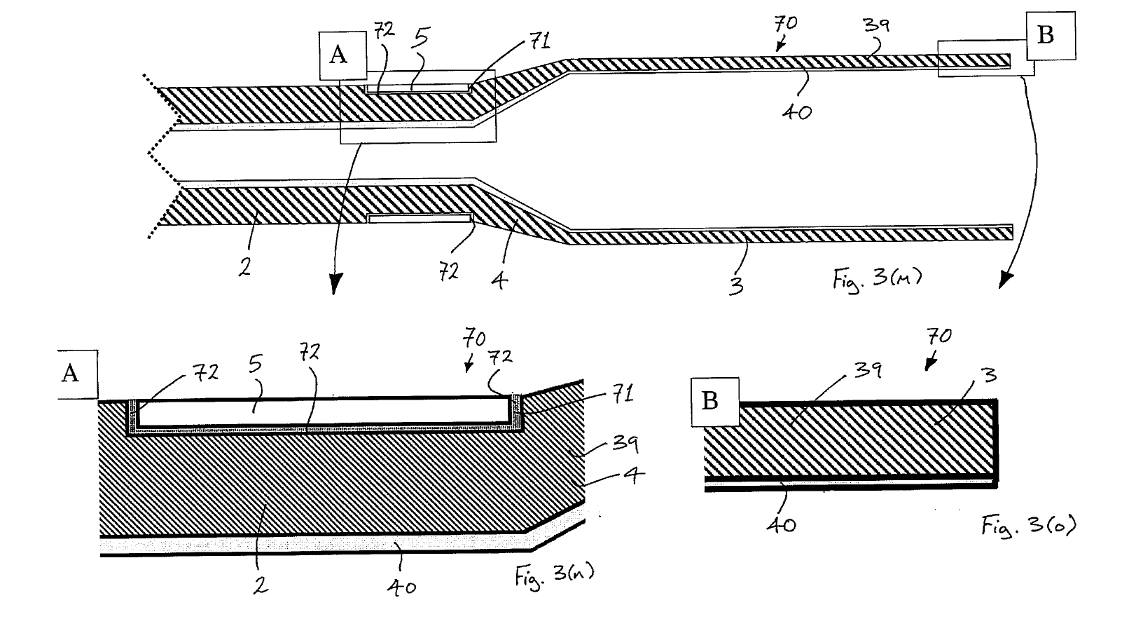

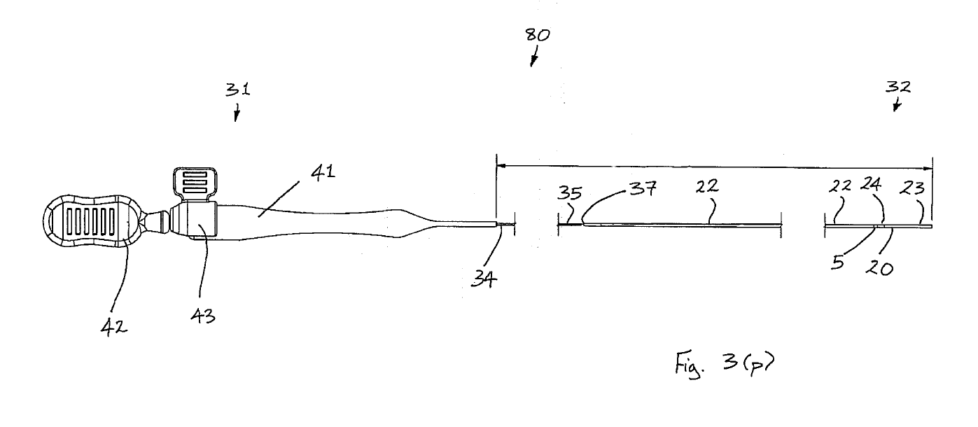

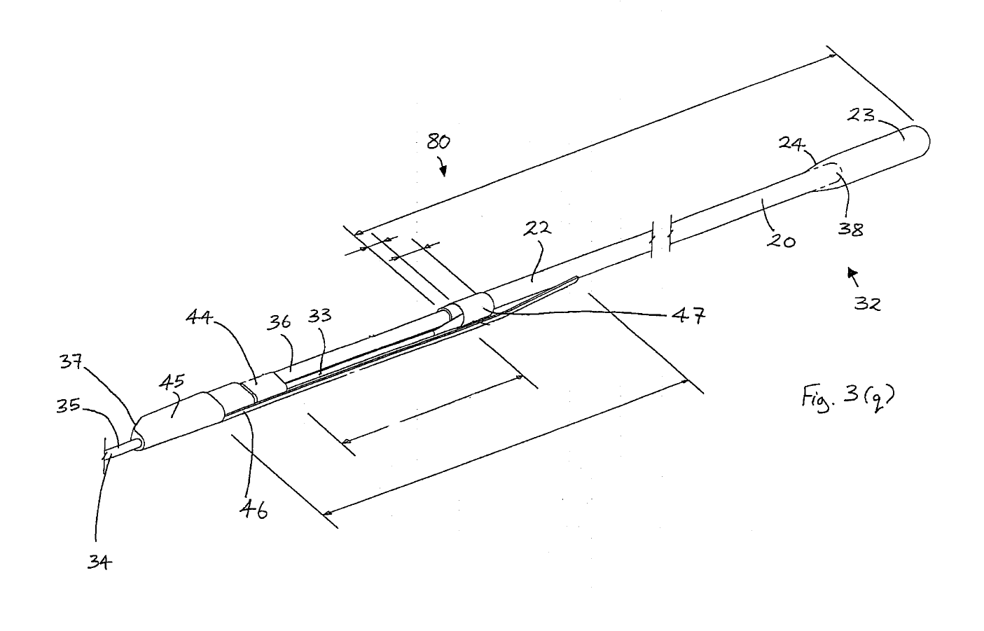

The pod 1 is flared outwardly and is necked to a smaller wall thickness (FIG. 3). In particular the pod 1 has a proximal portion 2, a distal portion 3, and a flared portion 4 which is intermediate the proximal portion 2 and the distal portion 3.

The pod 1 comprises a strengthening layer 39 of an intractable material, in this case of polyamide, and a low coefficient of friction material layer 40, in this case of polytetrafluoroethylene. The low-friction layer 40 is located radially inwardly of the strengthening layer 39. Each layer 39, 40 is integrally formed, and each layer 39, 40 extends from the proximal portion 2 to the distal portion 3.

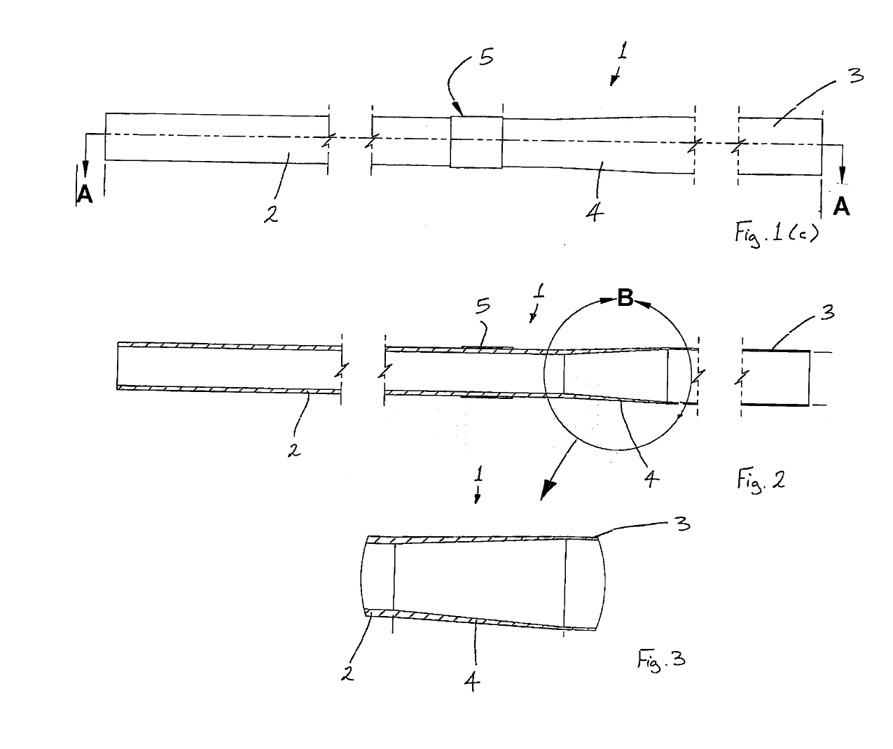

The outer diameter, the inner diameter and the wall thickness of the pod 1 are constant along the proximal portion 2 (FIG. 2). In particular, the outer diameter, the inner diameter and the wall thickness of each layer 39, 40 are constant along the proximal portion 2. Similarly the outer diameter, the inner diameter and the wall thickness of the pod 1 are constant along the distal portion 3 (FIG. 2). In particular the outer diameter, the inner diameter and the wall thickness of each layer 39, 40 are constant along the distal portion 3.

The intermediate portion 4 is flared, such that along the intermediate portion 4, the outer diameter and the inner diameter of the pod 1 increase gradually from the proximal portion 2 to the distal portion 3 (FIG. 3). In particular, the outer diameter and the inner diameter of each layer 39, 40 increase gradually from the proximal portion 2 to the distal portion 3. In addition, the intermediate portion 4 is necked such that along the intermediate portion 4, the wall thickness of the pod 1 decreases gradually from the proximal portion 2 to the distal portion 3 (FIG. 3). In particular the wall thickness of each layer 39, 40 decreases gradually from the proximal portion 2 to the distal portion 3.

The pod 1 is thin-walled. The wall thickness of the distal portion 3 of the pod 1 may be in the range of from 0.0008″ to 0.0012″ and is preferably approximately 0.0011″. The wall thickness of the proximal portion 2 of the pod 1 may be in the range of from 0.00225″ to 0.00325″, and is preferably approximately 0.00275″. In this case the wall thickness of the distal portion 3 of the pod 1 is approximately 40% of the wall thickness of the proximal portion 2 of the pod 1.

The wall thickness of the strengthening layer 39 is greater than the wall thickness of the low-friction layer 40, in this case approximately 10 times greater. In particular along the proximal portion 2 of the pod 1, the wall thickness of the strengthening layer 39 is in the range of from 0.00225″ to 0.00275″ and is preferably approximately 0.0025″, and the wall thickness of the low-friction layer 40 is preferably approximately 0.00025″. And in particular along the distal portion 3 of the pod 1, the wall thickness of the strengthening layer 39 is preferably approximately 0.001″, and the wall-thickness of the low-friction layer 40 is preferably approximately 0.0001″.

The inner diameter of the distal portion 3 of the pod 1 may be approximately 0.0395″. The length of the pod 1 from the proximal end to the distal end may be in the range of from 261 mm to 263 mm. The length of the intermediate portion 4 of the pod 1 may be approximately 2 mm.

The pod 1 is particularly suitable for use with an embolic protection filter having an outer diameter when deployed of approximately 7 mm.

During delivery of the filter, the distal end 38 of the pusher 36 is spaced proximally of the distal end of the pod 1, and the proximal end of an inner tubular member of the filter is partially inserted into the flared portion 4 of the pod 1. This arrangement provides a bridge in stiffness between the relatively stiff proximal portion 2 of the pod 1 and the relatively stiff inner tubular member of the filter. Thus the possibility of buckling of the relatively flexible distal portion 3 of the pod 1 is minimised.

The distal end 38 of the pusher 36 is engagable with the inner tubular member of the filter upon retraction of the pod 1 to deploy the filter out of the reception space (FIG. 3(i)).

The delivery catheter 30 also comprises a marker band 5 of a radiopaque material. The marker band 5 is mounted to the exterior surface of the proximal portion 2 of the pod 1, for example by means of bonding using an adhesive 102 (FIGS. 1(c) and 3(d)), to aid in visualisation of the pod 1 when the pod 1 is inserted into the vasculature of a patient.

The marker band 5 acts as a stop to limit movement of an embolic protection filter proximally relative to the pod 1, during loading of the filter into the pod 1.

The distance from the distal end of the marker band 5 to the distal end of the pod 1 may be in the range of from 20.5 mm to 21.5 mm. When the pod 1 is retracted to deploy the filter (FIG. 3(i)), the distance from the distal end of the marker band 5 to the distal end 38 of the pusher 36 may be in the range of from 32.5 mm to 34.5 mm.

The polytetrafluoroethylene (PTFE) layer 40 may be formed by extrusion. The pod 1 may be formed by dipping the solid PTFE layer 40 in a quantity of liquid polyimide.

During the process of manufacturing the delivery catheter 30, the intermediate portion 4 of the pod 1 is flared by inserting a flaring mandrel into the pod 1. The pod 1 is heated during the flaring process. The pod 1 is then necked by inserting a mandrel into the pod 1, locating emery paper on the exterior surface of the pod 1, and then engaging the pod 1 using a necking clamp with the emery paper between the pod 1 and the necking clamp. The pod 1 is heated during the necking process. The pod 1 is annealed after necking. The marker band 5 is then slid over the proximal end of the pod 1, and slid distally along the exterior surface of the pod 1 to the desired mounting position at the exterior surface of the proximal portion 2 of the pod 1.

In particular the 7 mm pod 1 or distal end of the polyimide catheter is flared outwardly to increase its internal diameter to house the 7 mm filter element. The marker band 5 is then advanced over the polyimide catheter from the proximal end and is bonded in place.

The process of manufacturing the delivery catheter 30 is illustrated in FIGS. 3(a) to 3(e). In further detail, the distal end of the pod 1 is flared to house the 7 mm filtration element. The flared pod 1 is then necked to reduce its wall thickness (FIG. 3(b)). The flare comprises an intermediate tapered section 4 of varying thickness and a distal straight section 3 of constant thickness. The marker band 5, with an internal diameter equal to the outer diameter of the polyimide tubing 2, is advanced from the proximal end of the polyimide tubing 1 until it meets the tapered lead to the flared pod.

The marker band 5 is adhesively bonded to the polyimide tubing 2 (FIG. 3(c)).

During the flaring procedure, the polyimide tubing is placed on a flaring mandrel. The polyimide tubing is heated as it is pushed over the flaring mandrel by heating blocks. The heating blocks move into position after the pod 1 is placed on the flaring mandrel. Once the pod 1 has passed through the heating blocks, a piece of emery paper is placed over the distal end of the flare and between the necking clamps. The flared pod 1 is then necked, with the flaring mandrel kept in place, to reduce the wall thickness of the pod 1 while maintaining the internal diameter determined by the flaring mandrel. The necked pod 1 is then annealed by leaving it under heat for 30 seconds. After the pod 1 is removed from the flaring mandrel, it is trimmed to the desired length.

The flaring process enables an ultra-thin layer 40 of PTFE to be achieved, as illustrated in FIG. 3(e).

At the proximal end 31 of the catheter 30, a distal handle 41 is provided for gripping the catheter body 34 and a proximal handle 42 is provided for gripping the actuator wire 33. The distal handle 41 is injection moulded over the hypotube 35 and the proximal handle 42 is crimped to the proximal end of the wire 33.

The handles 41, 42 are movable relative to one another in a telescoping manner with the proximal handle 42 sliding within the distal handle 41. Movement of the handles 41, 42 is limited by means of stop means. Abutment of an outward annular protrusion on the proximal handle 42 against the proximal end of the distal handle 41 prevents further movement of the proximal handle 42 distally relative to the distal handle 41. Engagement of a shoulder on the proximal handle 42 with an inward annular protrusion on the distal handle 41 prevents further movement of the proximal handle 42 proximally relative to the distal handle 41.

A releasable safety clip 43 is provided to maintain the handles 41, 42 fixed relative to one another.

The usable length from the distal end 100 of the distal handle 41 to the distal end of the pod 1 may be in the range of from 1345 mm to 1351 mm.

When the proximal handle 42 is moved to the distal-most position relative to the distal handle 41, the distance from the distal end of the constraining sleeve 44 to the proximal end of the pod 1 may be in the range of from 38 mm to 42 mm.

When the catheter 30 is assembled, the pod 1 is directly connected to the proximal handle 42, and the pusher 36 is directly connected to the distal handle 41. Movement of the proximal handle 42 proximally relative to the distal handle 41 moves the wire 33 and the pod 1 proximally relative to the pusher 36 to facilitate deployment of the filter from within the reception space.

The delivery catheter 30 may be used to deliver the embolic protection filter through a vasculature and to deploy the embolic protection filter downstream of a stenosed region in the vasculature to prevent potentially harmful emboli, which may be released into the blood stream during treatment of the stenosis, such as by a stenting procedure, from migrating further through the vascular system.

In use, a loading device is arranged along an external surface of the pod 1. A pushing device is then threaded through the tubular member of the filter and extended into the reception space. By moving the pushing device proximally, an engagement stop on the pushing device engages the distal end of the tubular member and the filter is moved towards the loading device. Continued proximal movement of the pushing device pushes the filter through the loading device, thereby collapsing the filter, and into the reception space. The marker band 5 acts as a stop to limit movement of the filter proximally relative to the pod 1.

The catheter 30 with the collapsed filter received within the reception space are then moved together proximally away from the loading device.

Next the guidewire is inserted into a vasculature and advanced through the vasculature until the guidewire has crossed a site of interest in the vasculature. A typical site of interest is a stenosed or diseased region of the vasculature. The delivery catheter 30 is then threaded over the guidewire by inserting the proximal end of the guidewire into the guidewire lumen at the distal end 38 of the pusher 36, through the lumen, and out of the lumen through the proximal guidewire opening 37. The catheter 30 is advanced over the guidewire in a rapid-exchange manner until the reception space is located downstream of the stenosis.

To deploy the filter at the desired site in the vasculature downstream of the stenosis, the proximal handle 42 is moved proximally while holding the distal handle 41 fixed, thereby causing the pull wire 33 and the pod 1 to be pulled proximally. The pod 1 moves proximally while the pusher 36 does not move. In this way, the collapsed filter is uncovered by the pod 1 while the distal end 38 of the pusher 36 abuts the proximal end of the tubular member of the filter. The delivery catheter 30 thus enables the self-expanding filter to expand outwardly to a deployed configuration. The distal end 38 of the pusher 36 acts as an abutment for a controlled, accurate deployment of the filter at the desired site in the vasculature.

When the filter has been fully deployed at the desired site in the vasculature, the delivery catheter 30 is withdrawn from the vasculature over the guidewire in a rapid-exchange manner to leave the deployed filter in place in the vasculature.

The movement of the elongate wire 33 proximally relative to the pusher 36 does not occlude the proximal guidewire opening 37, or in any way interfere with passage of the guidewire through the guidewire lumen. Thus rapid exchange of the delivery catheter 30 over the guidewire is possible during deployment of the filter.

During this deployment action, the pod 1 slides proximally in a telescoping manner over the pusher 36. In this manner, the filter may be accurately deployed in a controlled manner without the overall crossing profile of the delivery catheter 30 being adversely effected. In particular, no bulging or accordioning of the catheter 30 occurs during the deployment action.

The stainless steel pull wire 33 has a high tensile strength, and thus provides a stretch resistant link between the proximal pull handle 42 and the pod 1 to facilitate accurate and recoil free deployment of the embolic protection filter from within the reception space.

The hypotube 35 and the spring pusher 36 give the delivery catheter 30 excellent pushability and trackability for delivery through the vasculature, and provide extremely high compression resistance to significantly prevent compression, and thereby enable a smooth and accurate deployment action.

When the delivery catheter 30 is used to deploy the embolic protection filter in this manner, the non-moving elements of the catheter 30 are the distal handle 41, the hypotube 35, and the pusher 36. The moving elements of the catheter 30 are the proximal handle 42, the wire 33, and the pod 1.

The delivery catheter 30 of the invention facilitates accurate and intuitive filter deployment. By simply holding the distal handle 41 in a fixed position relative to the guide catheter and retracting the contoured proximal handle 42, the pull-wire 33 retracts the pod 1 and the filter is deployed.

The use of the internal pull wire 33 to connect the pod 1 to the proximal handle 42 ensures that the filter can be easily and accurately deployed in a precise location in a controlled, steady manner. In particular, the hypotube 35 and the distal handle 41 do not have to move relative to the guide catheter during the deployment action.

By attaching the pull-wire 33 to the proximal portion 2 of the pod 1, this arrangement ensures that the tensile force is transmitted from the wire 33 to the pod 1 proximally of the distal portion 3 of the pod 1. Thus the possibility of the distal portion 3 of the pod 1 being pulled to one side during wire retraction is minimised. Instead the pod 1 slides smoothly in the longitudinal direction for accurate filter deployment.

It will be appreciated that the filter may be deployed by any suitable movement of the pod 1 proximally relative to the pusher 36. For example the pusher 36 may be advanced distally while maintaining the position of the pod 1 fixed to deploy the filter from within the reception space.

The rapid exchange delivery catheter 30 of the invention facilitates the delivery of a bare wire filtration element over a standard length (180˜190 cm) stepped guidewire.

In FIGS. 3(j) to 3(l) there is illustrated a distal pod 60 of another delivery catheter according to the invention, which is similar to the pod 1 of FIGS. 1(a) to 3(i), and similar elements in FIGS. 3(j) to 3(l) are assigned the same reference numerals.

In this case, the exterior surface of the proximal portion 2 of the pod 60 has a mounting recess 61 into which the visualisation element 5 is mounted and then bonded to the exterior surface of the pod 60, for example using an adhesive 102.

FIGS. 3(m) to 3(o) illustrate a distal pod 70 of a further delivery catheter according to the invention, which is similar to the pod 60 of FIGS. 3(j) to 3(l), and similar elements in FIGS. 3(m) to 3(o) are assigned the same reference numerals.

In this case the mounting recess 71 is deeper than the mounting recess 61 of FIGS. 3(j) to 3(l). When the visualisation element 5 is mounted into the mounting recess 71, the exterior surface of the visualisation element 5 is flush with the exterior surface of the pod 70 on either side of the mounting recess 71 (FIG. 3(n)). The visualisation element 5 is then bonded to the exterior surface of the pod 70 in the mounting recess 71 by means of adhesive 72.

The recessed marker band 5 removes abrupt edges which might otherwise damage tissue during advancement of the delivery catheter.

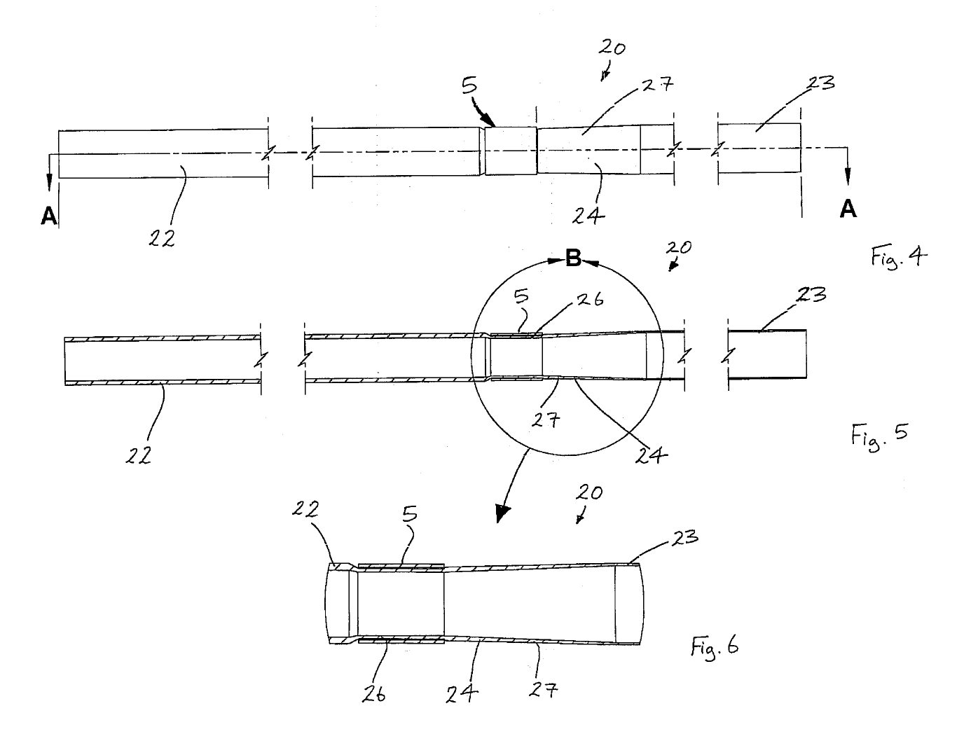

In FIGS. 3(p) to 6(k) there is illustrated another delivery catheter 80 according to the invention, which is similar to the catheter 30 of FIGS. 1(a) to 3(i), and similar elements in FIGS. 3(p) to 6(k) are assigned the same reference numerals.

In this case the distal pod 20 has a proximal portion 22, a distal portion 23 and an intermediate portion 24. The intermediate portion 24 has a proximal necked portion 26 and a distal flared portion 27.

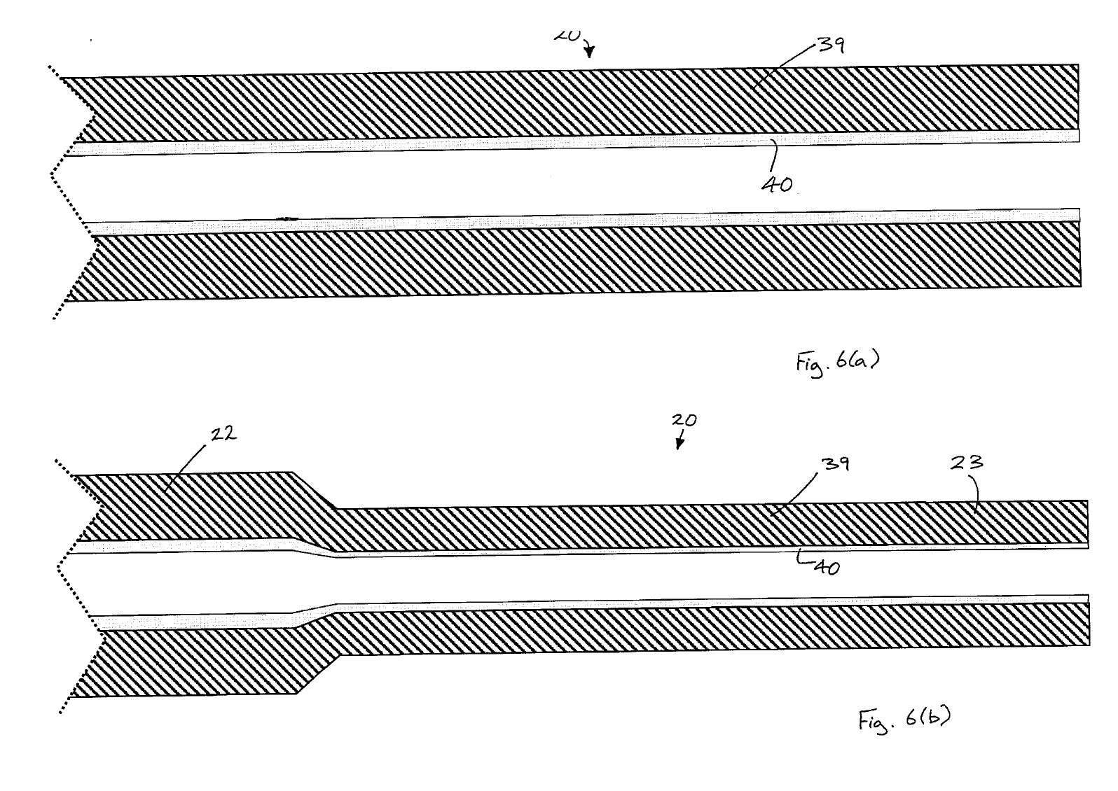

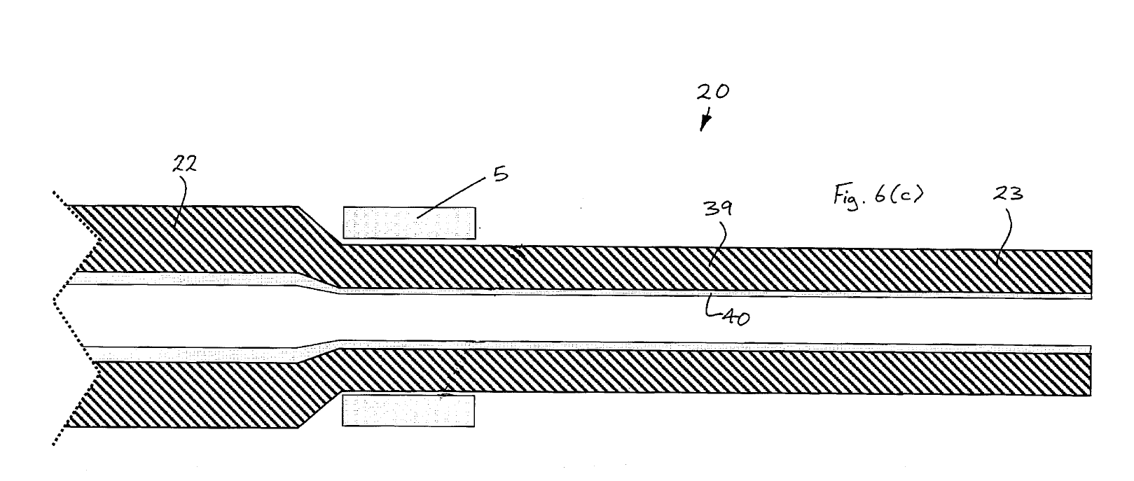

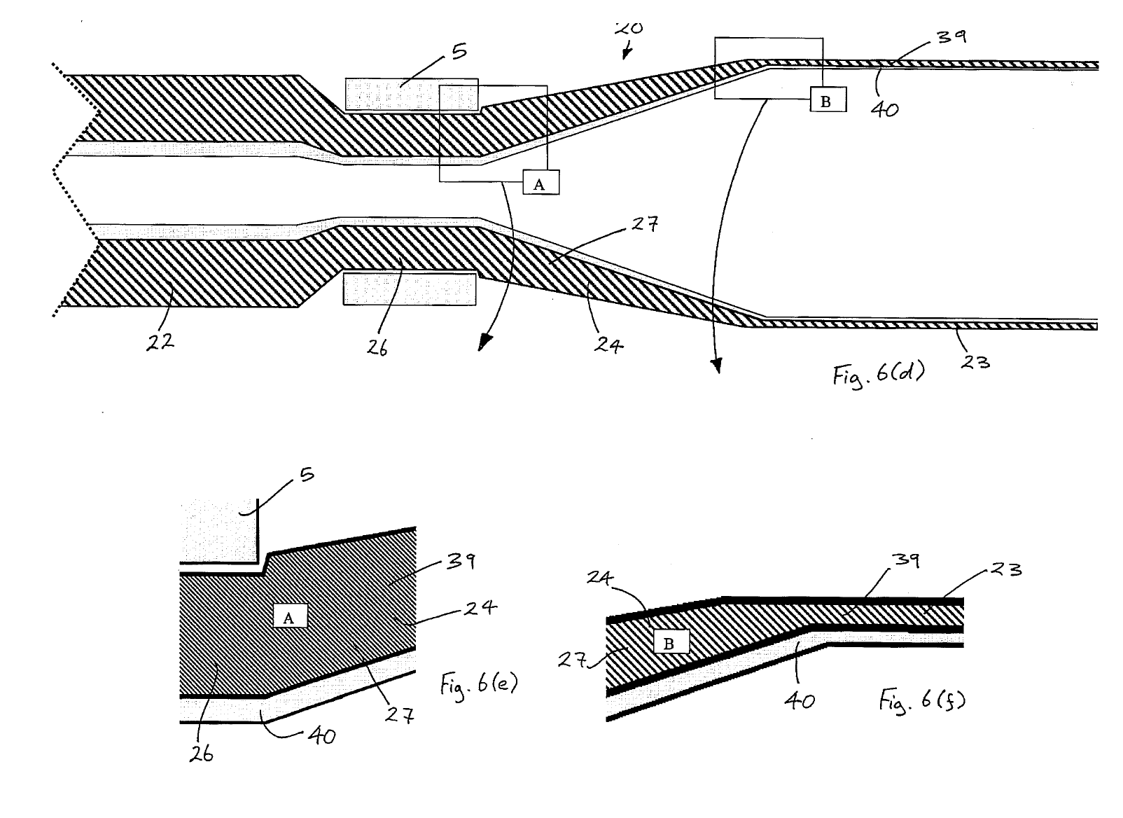

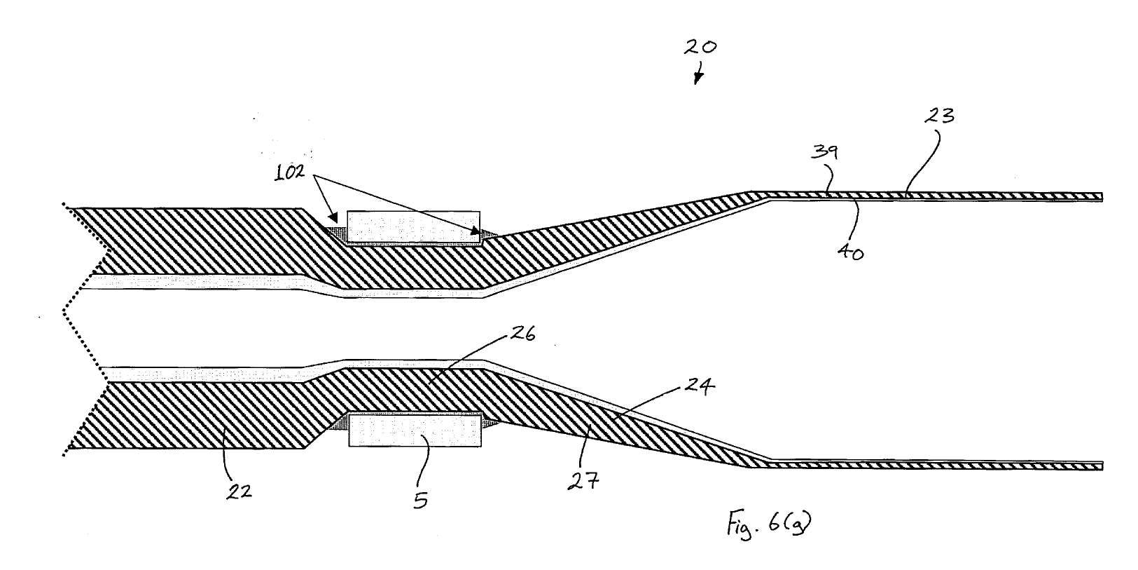

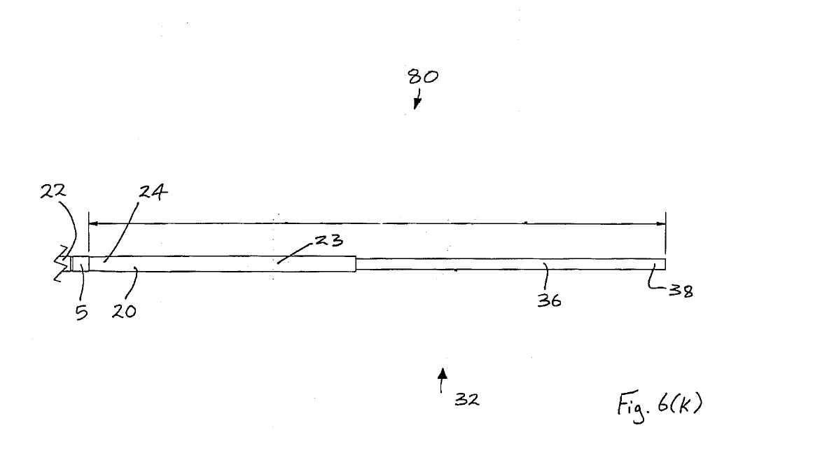

Along the necked portion 26, the outer diameter, the inner diameter and the wall thickness of the pod 20 are constant (FIG. 6(d)). In particular the outer diameter, the inner diameter and the wall thickness of each layer 39, 40 are constant along the necked portion 26. In addition the outer diameter of each layer 39, 40 of the proximal portion 22 is greater than the outer diameter of each corresponding layer 39, 40 of the necked portion 26, the inner diameter of each layer 39, 40 of the proximal portion 22 is greater than the inner diameter of each corresponding layer 39, 40 of the necked portion 26, and the wall thickness of each layer 39, 40 of the proximal portion 22 is greater than the wall thickness of each corresponding layer 39, 40 of the necked portion 26 (FIG. 6(d)).

Along the flared portion 27, the outer diameter and the inner diameter of the pod 20 increase from the necked portion 26 to the distal portion 23 (FIG. 6(d)). In particular, the outer diameter and the inner diameter of each layer 39, 40 increase from the necked portion 26 to the distal portion 23. In addition, along the flared portion 27, the wall thickness of the pod 20 decreases from the necked portion 26 to the distal portion 23 (FIG. 6(d)). In particular the wall thickness of each layer 39, 40 decreases from the necked portion 26 to the distal portion 23.

The inner diameter of the distal portion 23 of the pod 20 may be approximately 0.0345″. The pod 20 is particularly suitable for use with an embolic protection filter having an outer diameter when deployed of approximately 5 mm.

The marker band 5 is mounted to the exterior surface of the necked portion 26 of the pod 20 (FIG. 6(d)). The distance from the distal end of the marker band 5 to the distal end of the pod 1 may be in the range of from 15 mm to 16 mm.

During the process of manufacturing the delivery catheter 80, the portion 26 of the pod 20 is necked by inserting a mandrel into the pod 20, locating emery paper on the exterior surface of the pod 20, and then engaging the pod 20 using a necking clamp with the emery paper between the pod 20 and the necking clamp. The pod 20 is heated during the necking process. The marker band 5 is then slid over the distal end of the pod 20, and slid proximally along the exterior surface of the pod 20 to the desired mounting position at the exterior surface of the necked portion 26 of the pod 20. The portion 27 is then flared by inserting a flaring mandrel into the pod 20 from the distal end. The pod 20 is heated during the flaring process.

The necking process reduces the wall thickness of the pod 1 while maintaining the internal diameter of the pod 1.

In particular the 5 mm pod 20 or distal end of the polyimide catheter is preformed. This preforming step effectively necks the distal end 26 of the catheter so that its internal and outer diameters are reduced while also decreasing the wall thickness. The marker band 5 has an internal diameter equal to the outer diameter of the necked distal end 26 of the polyimide catheter and an outer diameter equal to the outer diameter of the proximal end 22 of the polyimide catheter. The marker band 5 is advanced over the polyimide tubing 20 from the distal end of the catheter pod 20 until it forms an abutment with the necked end 26 of the catheter pod 20. The distal end 27 of the polyimide catheter, distal to the marker band 5, is then flared to accommodate the 5 mm filter element. The marker band 5 is then bonded to the polyimide catheter 26 in place. These extra steps for the 5 mm catheter 20 facilitate in keeping the marker band outer diameter inline with that of the proximal portion 22 of the pod 20.

The marker band 5 maintains the pre-formed shape of the necked portion 26 of the pod 20 during the flaring process, as illustrated in FIG. 6(e). There is a step-up raise in outer diameter of the pod 20 at the distal end corner of the marker band 5 (FIG. 6(e)).

As illustrated in FIG. 6(f), the pod 20 is necked with the flaring mandrel remaining in place to reduce the profile while maintaining the internal diameter.

Adhesive 102 is used to secure the marker band 5 to the exterior surface of the pod 20 (FIG. 6(g)).

With reference to the process of manufacturing the delivery catheter 80, the distal end of the pod 20 is necked down to have an outer diameter equal to the inner diameter of the marker band 5 (FIG. 6(b)). The neck comprises a tapered section of varying thickness and a straight section 26 of constant thickness. The marker band 5, with an outer diameter equal to the outer diameter of the un-necked polyimide tubing 22, is advanced from the distal end of the necked down polyimide tubing until it meets the tapered lead to the un-necked polyimide tubing (FIG. 6(c)). The pod distal to the marker band 5 is then flared to house a 5 mm filtration element. The flared pod is then necked to reduce its wall thickness (FIG. 6(d)). The flare comprises a tapered section 27 of varying thickness and a distal straight section 23 of constant thickness. The procedure for flaring the 5 mm pod 20 is similar to that described previously with reference to the 7 mm pod 1 of FIGS. 1(a) to 3(i). The marker band 5 is adhesively bonded in place after all forming steps are complete (FIG. 6(g)).

The preforming procedure reduces the outer diameter of the polyimide tubing 20 to allow the marker band 5 to be pushed back from the distal end of the pod 20. The polyimide tubing is placed on a necking mandrel. The polyimide tubing is gripped by the necking clamp using emery paper. The necking clamp advances forward reducing the diameter of the polyimide tubing to that of the necking mandrel. The heat blocks move into position and heat the polyimide tubing as the necking clamp advances forward. Once necked down, the pod 20 is annealed by keeping it under heat for 30 seconds.

The necking and flaring process enables an ultra-thin layer 40 of PTFE to be achieved, as illustrated in FIG. 6(d).

The delivery catheter of the invention is also suitable for over-the-wire exchange over a guidewire. The rapid exchange configuration is not essential.

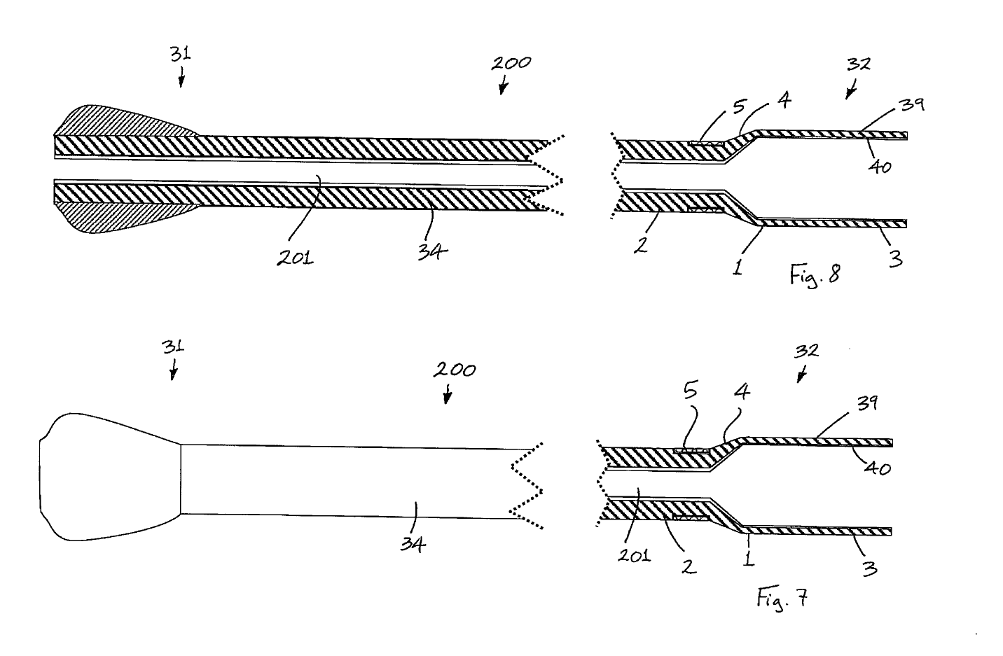

Referring to FIGS. 7 and 8 there is illustrated another delivery catheter 200 according to the invention, which is similar to the delivery catheter 30 of FIGS. 1(a) to 3(i), and similar elements in FIGS. 7 and 8 are assigned the same reference numerals.

In this case the pod 1 is fixedly attached to the distal end of the catheter body 34. The pod 1 is not movable relative to the catheter body 34.

The guidewire lumen 201 extends through the catheter body 34 from the proximal end of the catheter body 34 to the distal end of the pod 1. The catheter 200 is thus suitable for over-the-wire exchange over a guidewire.

FIGS. 7 and 8 illustrate the over the wire sheath 34.

The catheter 200 is suitable for delivery and deployment of an embolic protection filter which is coupled to a guidewire. The filter may be deployed from the reception space by moving the catheter 200 proximally relative to the guidewire.

The catheter 200 is suitable for delivery and deployment of an embolic protection filter having an outer diameter when deployed of approximately 7 mm.

In FIGS. 9 and 10 there is illustrated another delivery catheter 210 according to the invention, which is similar to the delivery catheter 200 of FIGS. 7 and 8, and similar elements in FIGS. 9 and 10 are assigned the same reference numerals.

In this case the catheter 210 is suitable for delivery and deployment of an embolic protection filter having an outer diameter when deployed of approximately 5 mm.

FIGS. 9 and 10 illustrate the over the wire sheath 34.

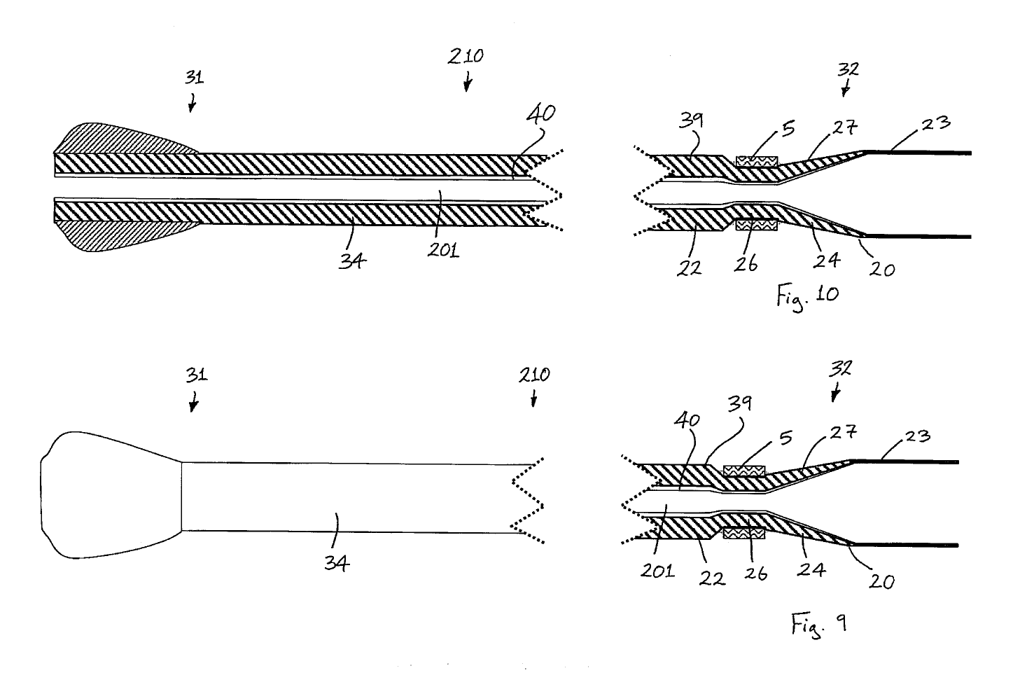

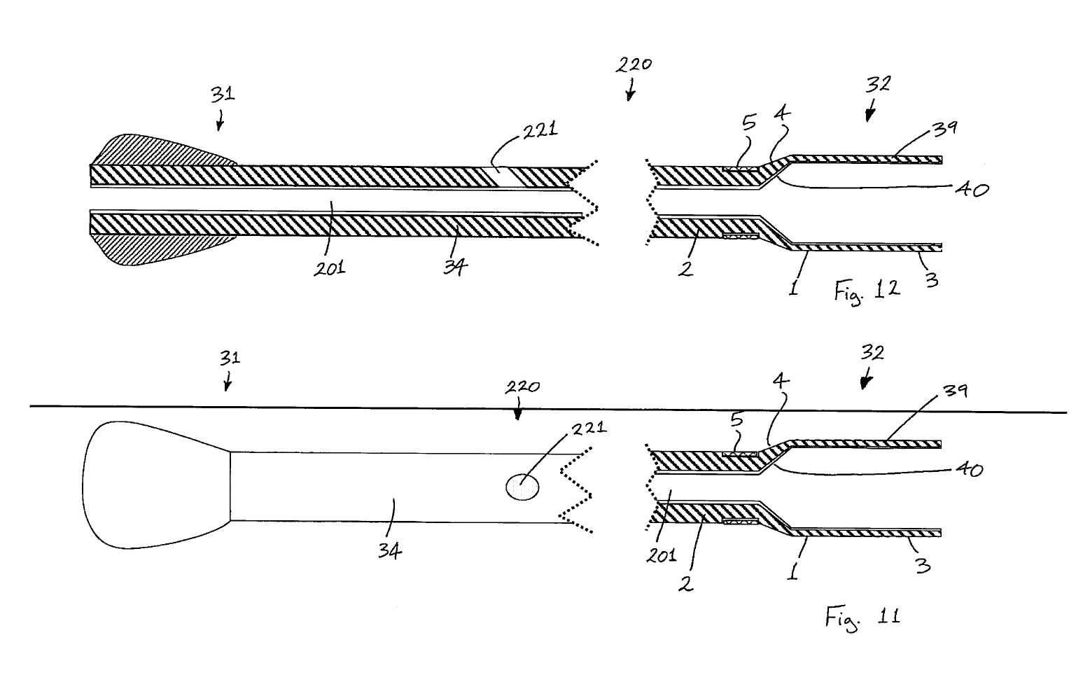

FIGS. 11 and 12 illustrate another delivery catheter 220 according to the invention, which is similar to the delivery catheter 200 of FIGS. 7 and 8, and similar elements in FIGS. 11 and 12 are assigned the same reference numerals.

In this case the guidewire lumen 201 extends through the catheter body 34 from a proximal rapid exchange guidewire opening 221 in a sidewall of the catheter body 34 to the distal end of the pod 1. The catheter 220 is thus suitable for rapid exchange over a guidewire.

FIGS. 11 and 12 illustrate the hole in the wall sheath 34.

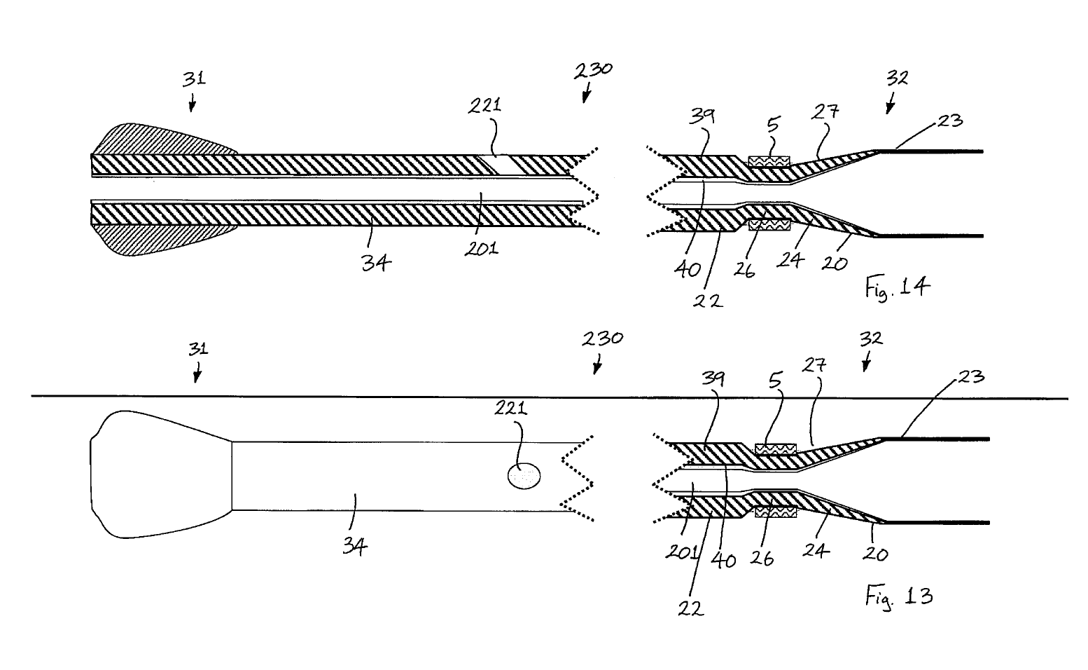

Referring to FIGS. 13 and 14 there is illustrated another delivery catheter 230 according to the invention which is similar to the delivery catheter 210 of FIGS. 9 and 10, and similar elements in FIGS. 13 and 14 are assigned the same reference numerals.

In this case the guidewire lumen 201 extends through the catheter body 34 from the proximal rapid exchange guidewire opening 221 in the sidewall of the catheter body 34 in the distal end of the pod 20. The catheter 230 is thus suitable for rapid exchange over a guidewire.

FIGS. 13 and 14 illustrate the hole in the wall sheath 34.

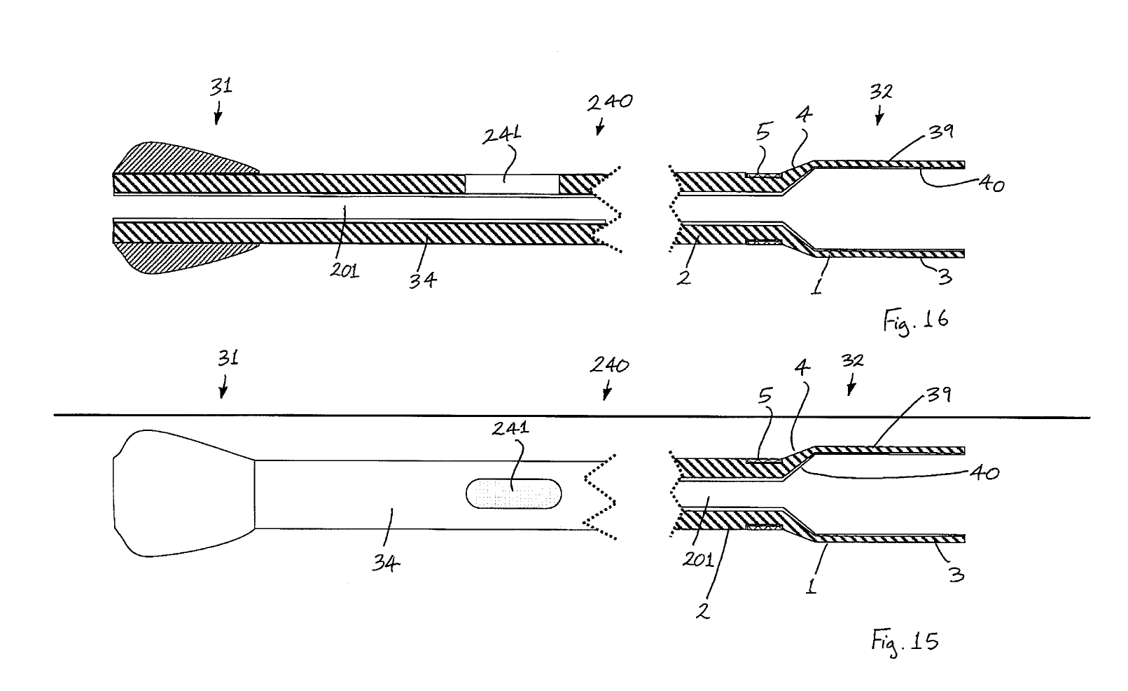

In FIGS. 15 and 16 there is illustrated another delivery catheter 240 according to the invention, which is similar to the delivery catheter 220 of FIGS. 11 and 12, and similar elements in FIGS. 15 and 16 are assigned the same reference numerals.

In this case the proximal guidewire opening 241 is substantially elongate.

FIGS. 17 and 18 illustrate another delivery catheter 250 according to the invention, which is similar to the delivery catheter 230 of FIGS. 13 and 14, and similar elements in FIGS. 17 and 18 are assigned the same reference numerals.

In this case the proximal guidewire opening 241 is substantially elongate.

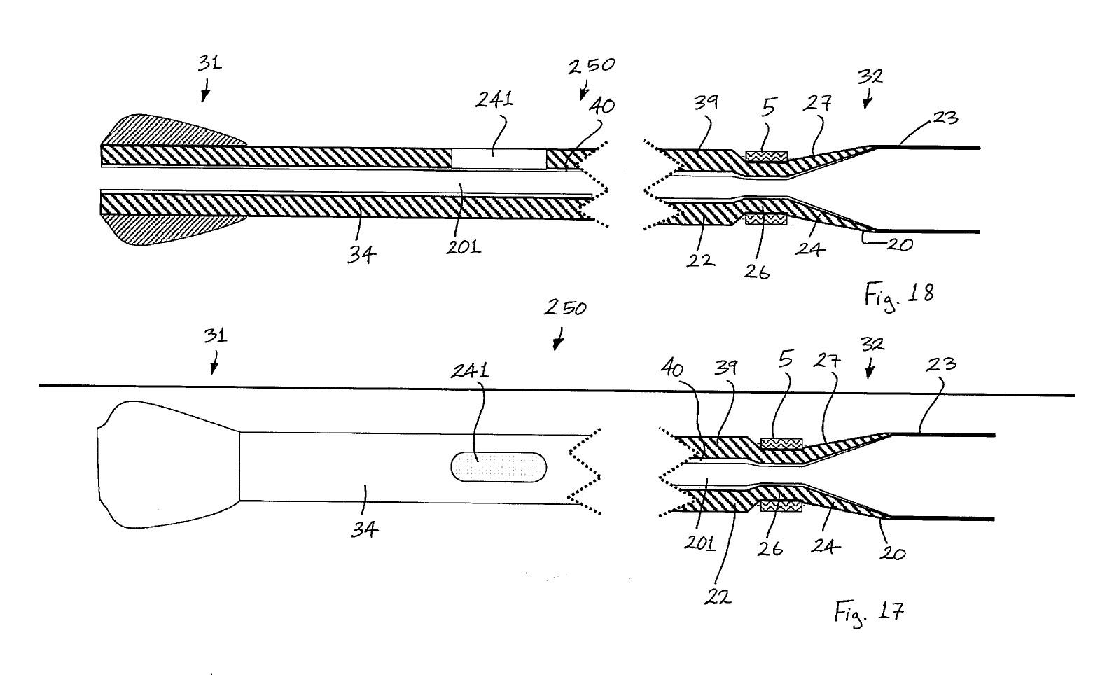

Referring to FIGS. 19 and 20 there is illustrated another delivery catheter 260 according to the invention, which is similar to the delivery catheter 220 of FIGS. 11 and 12, and similar elements in FIGS. 19 and 20 are assigned the same reference numerals.

In this case the catheter body 34 comprises an elongate slit 261 extending proximally from the proximal guidewire opening 221 to the proximal end of the catheter body 34.

FIGS. 19 and 20 illustrate the split sheath 34.

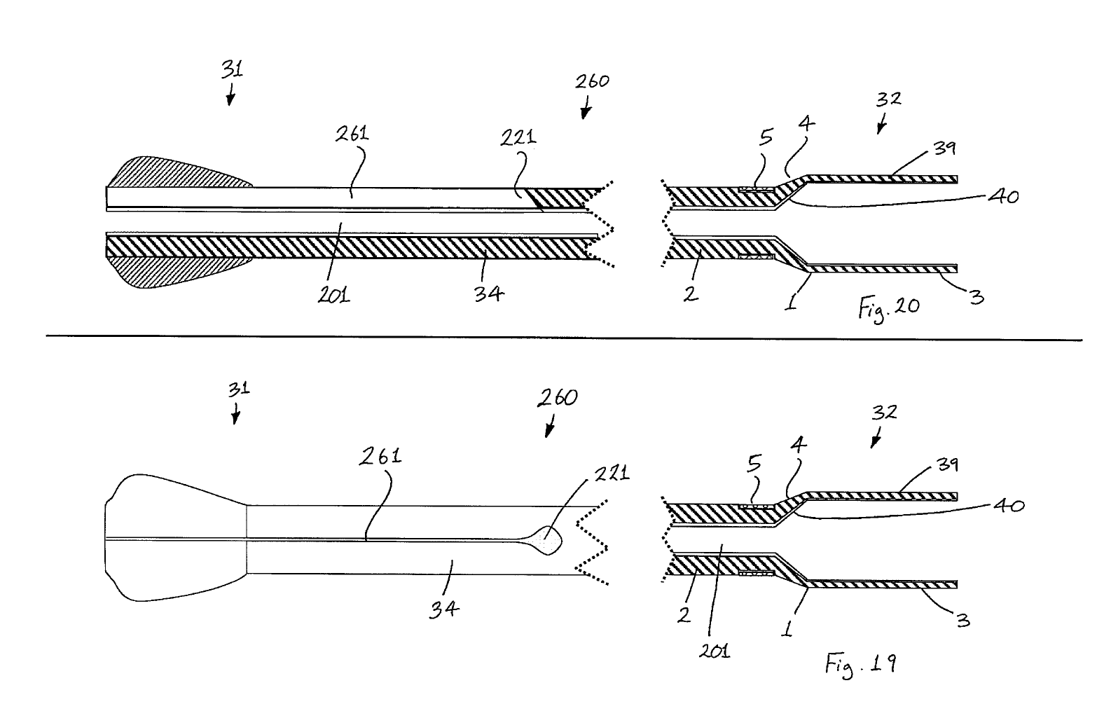

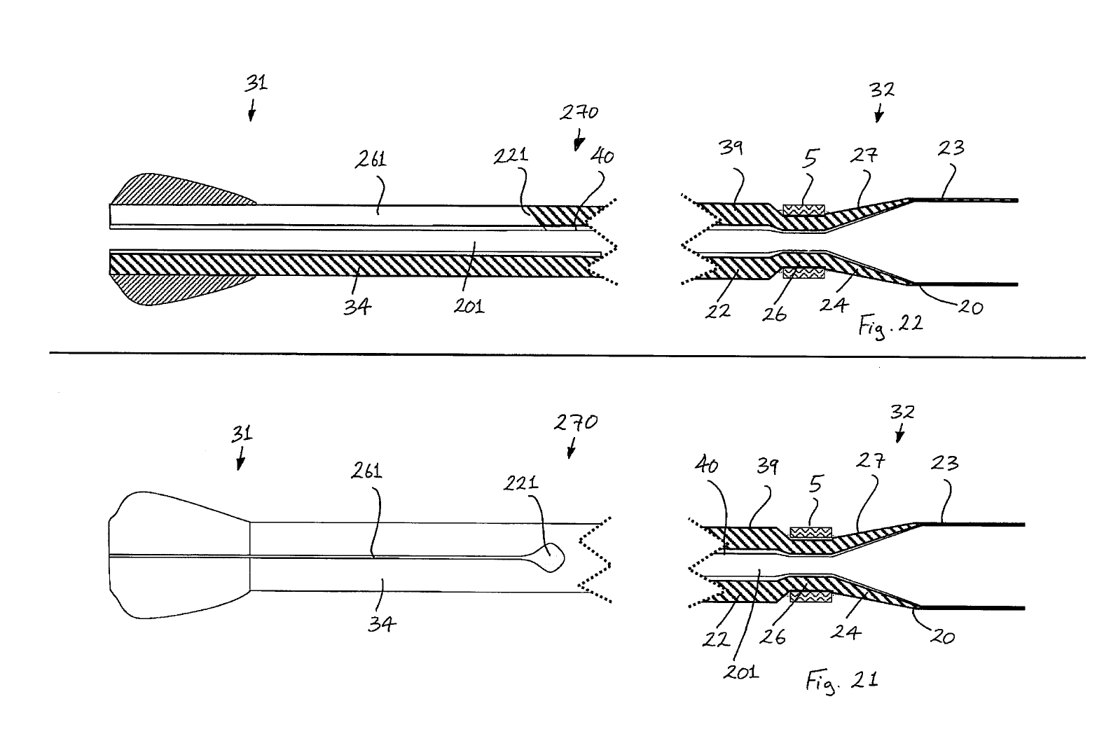

In FIGS. 21 and 22 there is illustrated another delivery catheter 270 according to the invention, which is similar to the delivery catheter 230 of FIGS. 13 and 14, and similar elements in FIGS. 21 and 22 are assigned the same reference numerals.

In this case the catheter body 34 comprises the elongate slit 261 extending proximally from the proximal guidewire opening 221 to the proximal end of the catheter body 34.

FIGS. 21 and 22 illustrate the split sheath 34.

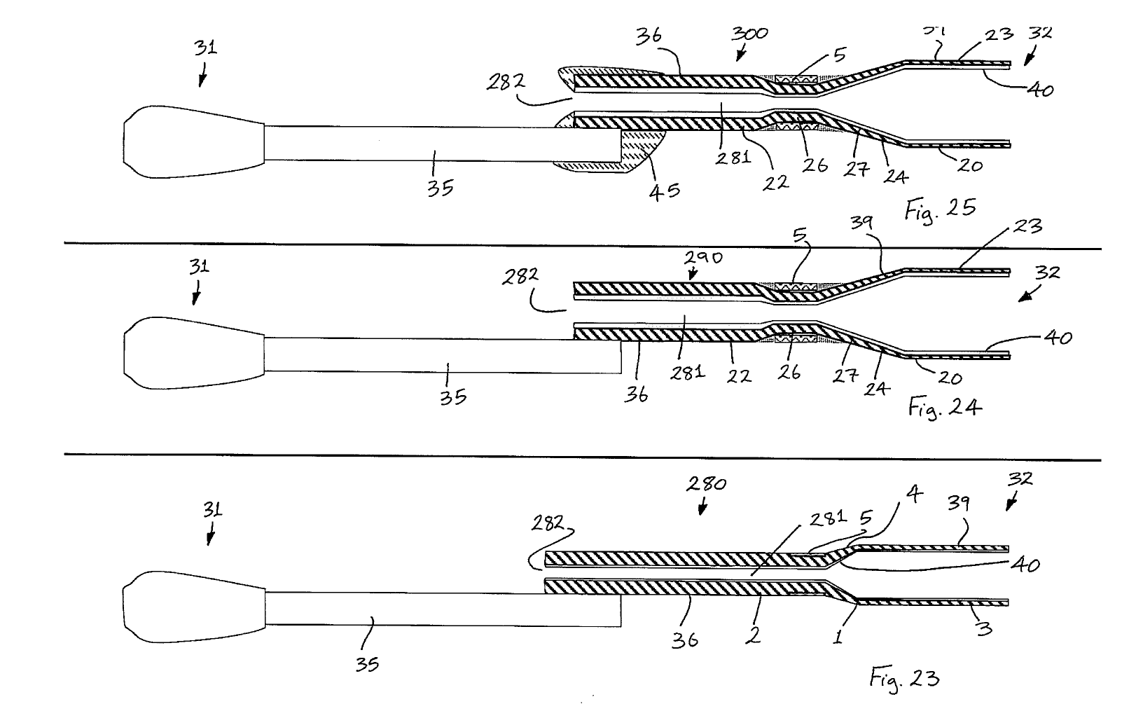

FIG. 23 illustrates a further delivery catheter 280 according to the invention, which is similar to the delivery catheter 220 of FIGS. 11 and 12, and similar elements in FIG. 23 are assigned the same reference numerals.

In this case the catheter body 34 comprises the catheter shaft 35 and the engagement element 36. The catheter shaft 35 is fixedly attached to the engagement element 36 in a side-by-side overlapping arrangement.

The guidewire lumen 281 extends through the engagement element 36 from the proximal rapid exchange guidewire opening 282 to the distal end of the pod 1.

FIG. 23 illustrates the over the wire sheath 34.

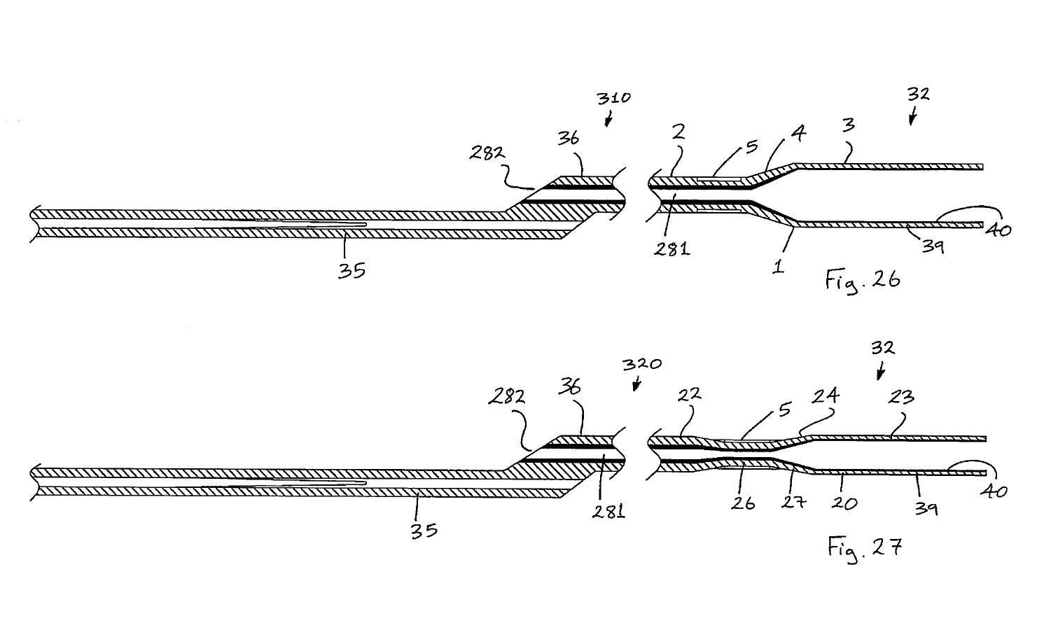

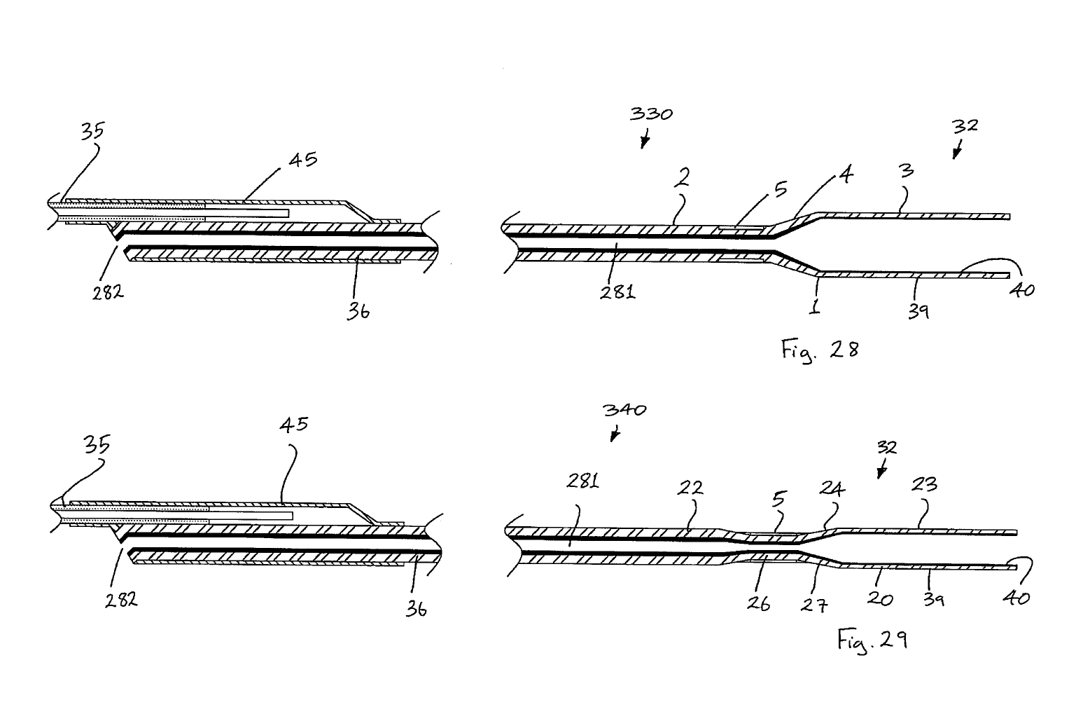

Referring to FIG. 24 there is illustrated a further delivery catheter 290 according to the invention, which is similar to the delivery catheter 230 of FIGS. 13 and 14, and similar elements in FIG. 24 are assigned the same reference numerals.

In this case the catheter body 34 comprises the catheter shaft 35 and the engagement element 36. The catheter shaft 35 is fixedly attached to the engagement element 36 in a side-by-side overlapping arrangement.

The guidewire lumen 281 extends through the engagement element 36 from the proximal rapid exchange guidewire opening 282 to the distal end of the pod 20.

In FIG. 25 there is illustrated another delivery catheter 300 according to the invention, which is similar to the delivery catheter 290 of FIG. 24, and similar elements in FIG. 25 are assigned the same reference numerals.

In this case the catheter shaft 35 is fixedly attached to the engagement element 36 by means of the overmould joiner 45.

FIG. 26 illustrates another delivery catheter 310 according to the invention, which is similar to the delivery catheter 280 of FIG. 23, and similar elements in FIG. 26 are assigned the same reference numerals.

In this case the catheter shaft 35 is formed integrally with the engagement element 36.

Referring to FIG. 27 there is illustrated another delivery catheter 320 according to the invention, which is similar to the delivery catheter 290 of FIG. 24, and similar elements in FIG. 27 are assigned the same reference numerals.

In this case the catheter shaft 35 is formed integrally with the engagement element 36.

In FIG. 28 there is illustrated another delivery catheter 330 according to the invention, which is similar to the delivery catheter 280 of FIG. 23, and similar elements in FIG. 28 are assigned the same reference numerals.

In this case the catheter shaft 35 is fixedly attached to the engagement element 36 by means of the overmould joiner 45.

FIG. 29 illustrates a further delivery catheter 340 according to the invention, which is similar to the delivery catheter 300 of FIG. 25, and similar elements in FIG. 29 are assigned the same reference numerals.

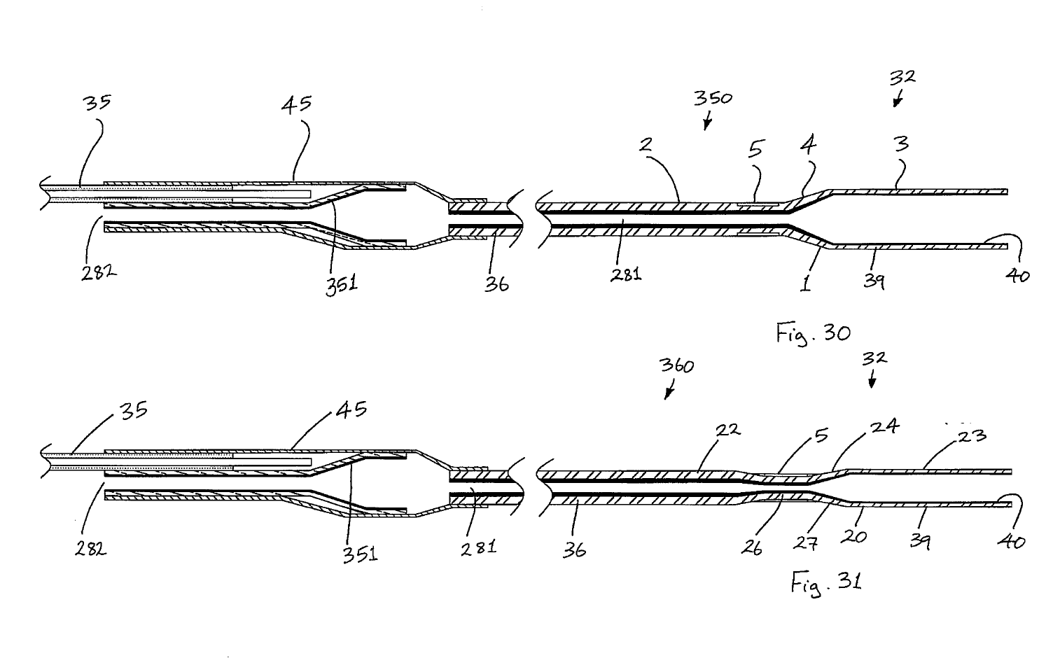

Referring to FIG. 30 there is illustrated another delivery catheter 350 according to the invention, which is similar to the delivery catheter 330 of FIG. 28, and similar elements in FIG. 30 are assigned the same reference numerals.

In this case the proximal end of the engagement element 36 is spaced distally of the distal end of the catheter shaft 35. A tapered tube 351 is provided to guide the guidewire out of the guidewire lumen 281 through the proximal guidewire opening 282.

In FIG. 31 there is illustrated another delivery catheter 360 according to the invention, which is similar to the delivery catheter 340 of FIG. 29, and similar elements in FIG. 31 are assigned the same reference numerals.

In this case the proximal end of the engagement element 36 is spaced distally of the distal end of the catheter shaft 35. A tapered tube 351 is provided to guide the guidewire out of the guidewire lumen 281 through the proximal guidewire opening 282.

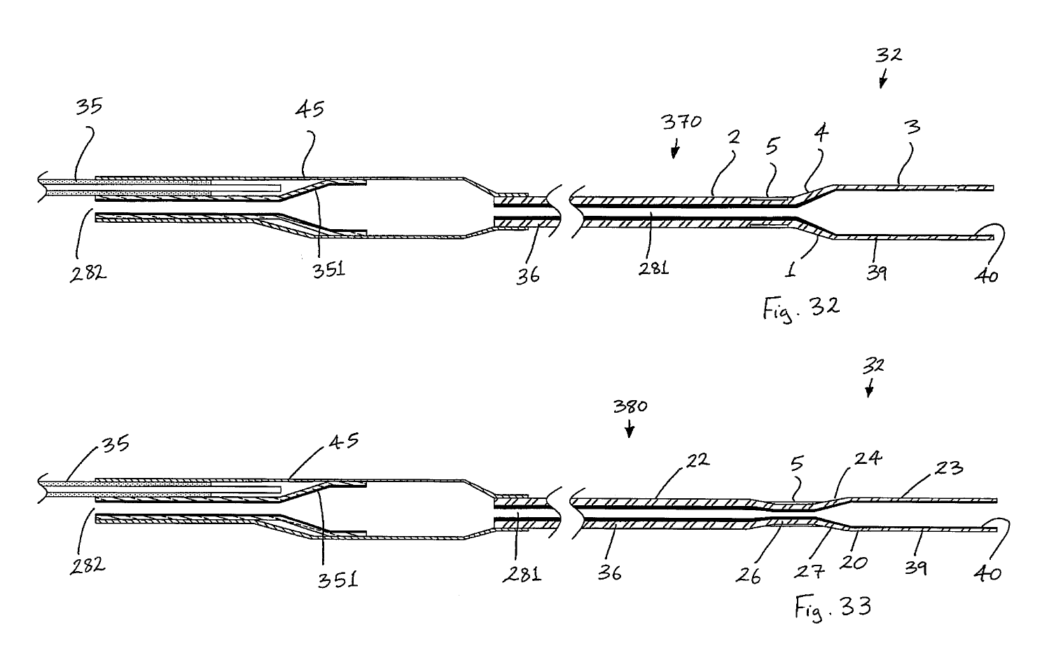

FIG. 32 illustrates a further delivery catheter 370 according to the invention, which is similar to the delivery catheter 350 of FIG. 30, and similar elements in FIG. 32 are assigned the same reference numerals.

In this case the proximal end of the engagement element 36 is spaced further distally from the distal end of the catheter shaft 35.

Referring to FIG. 33 there is illustrated another delivery catheter 380 according to the invention, which is similar to the delivery catheter 360 of FIG. 31, and similar elements in FIG. 33 are assigned the same reference numerals.

In this case the proximal end of the engagement element 36 is spaced further distally from the distal end of the catheter shaft 35.

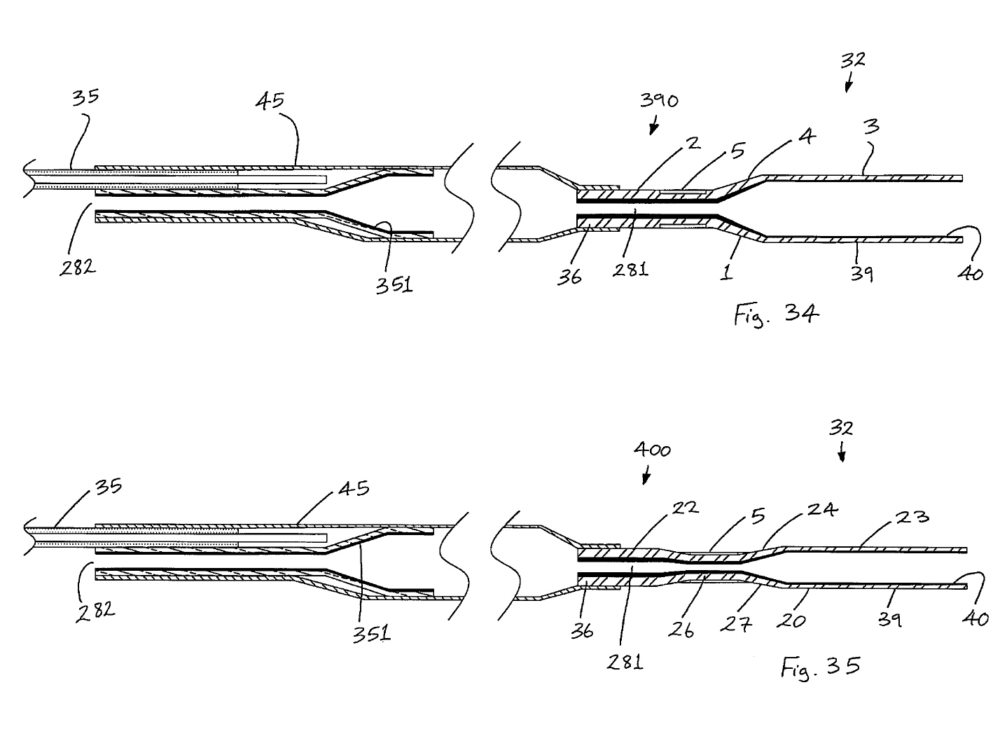

In FIG. 34 there is illustrated another delivery catheter 390 according to the invention, which is similar to the delivery catheter 370 of FIG. 32, and similar elements in FIG. 34 are assigned the same reference numerals.

In this case the proximal end of the engagement element 36 is spaced even further distally from the distal end of the catheter shaft 35.

FIG. 35 illustrates a further delivery catheter 400 according to the invention, which is similar to the delivery catheter 380 of FIG. 33, and similar elements in FIG. 35 are assigned the same reference numerals.

In this case the proximal end of the engagement element 36 is spaced even further distally from the distal end of the catheter shaft 35.

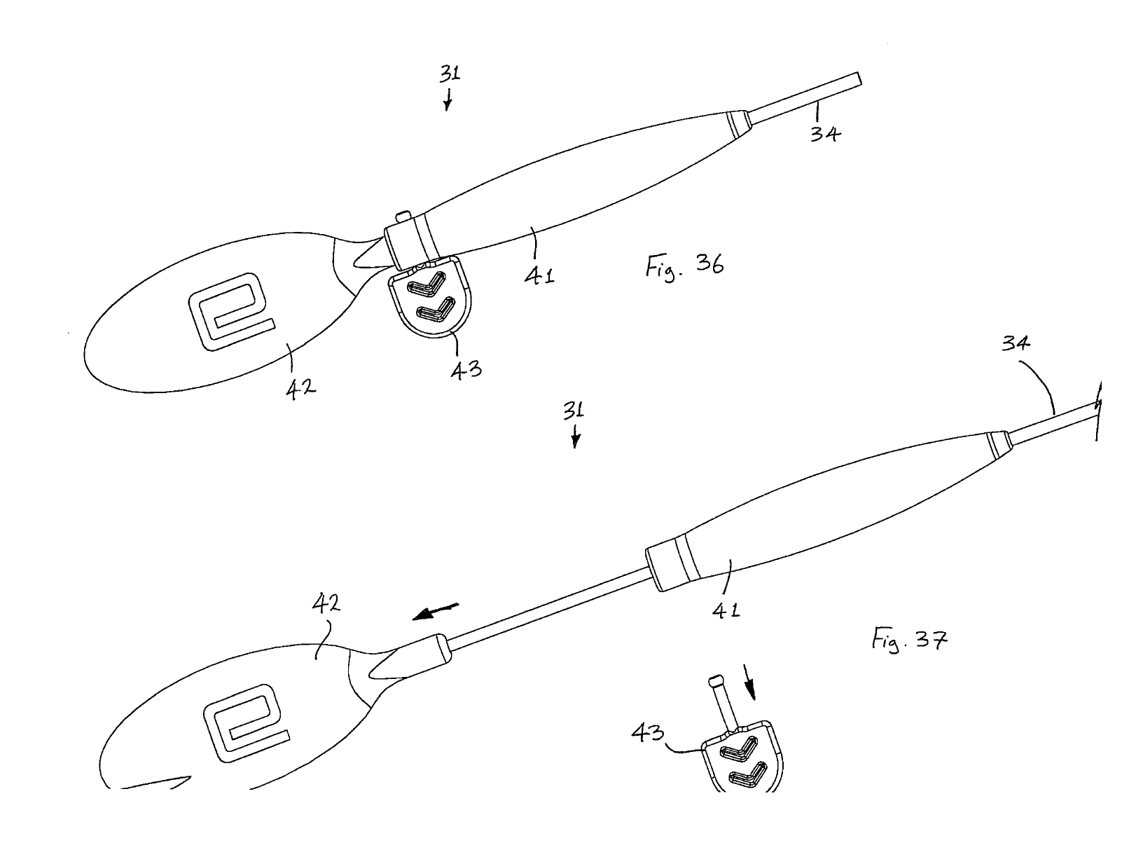

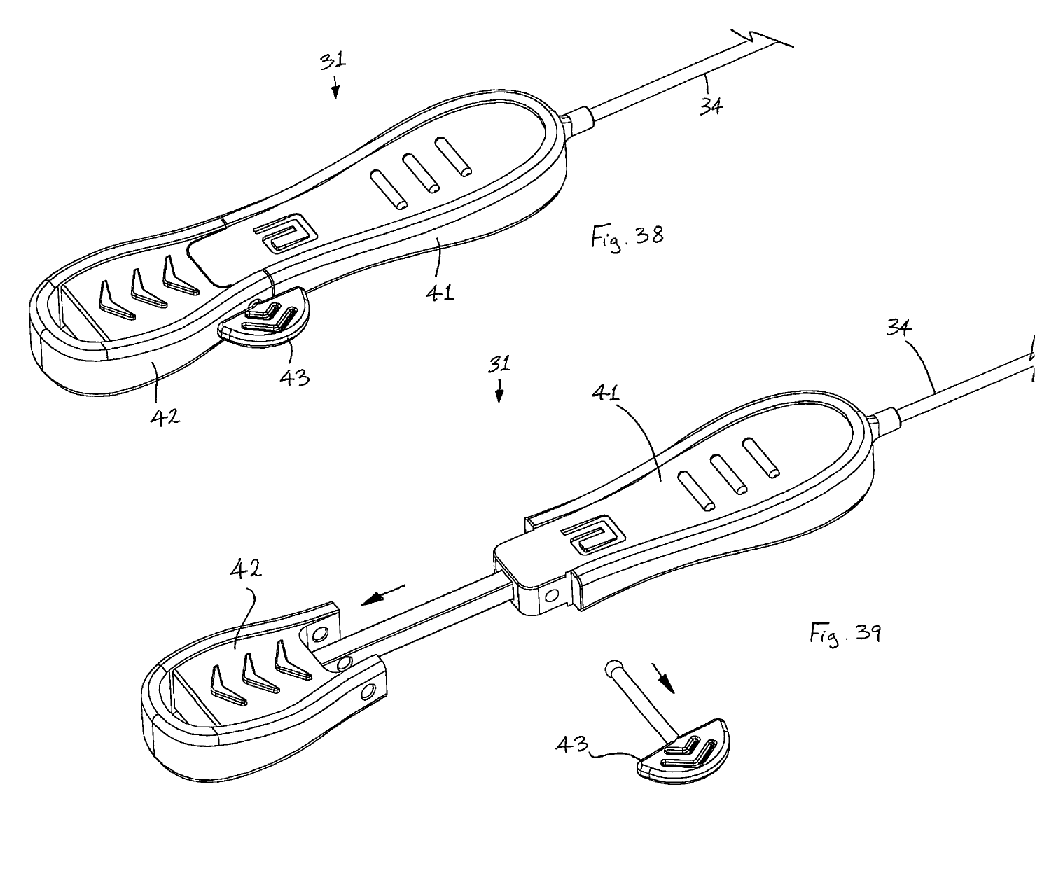

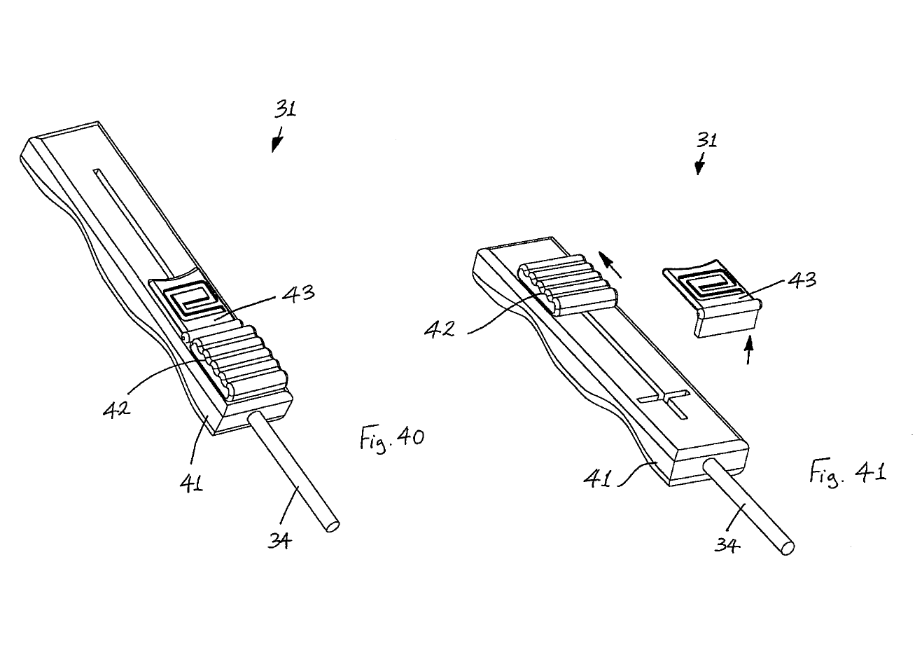

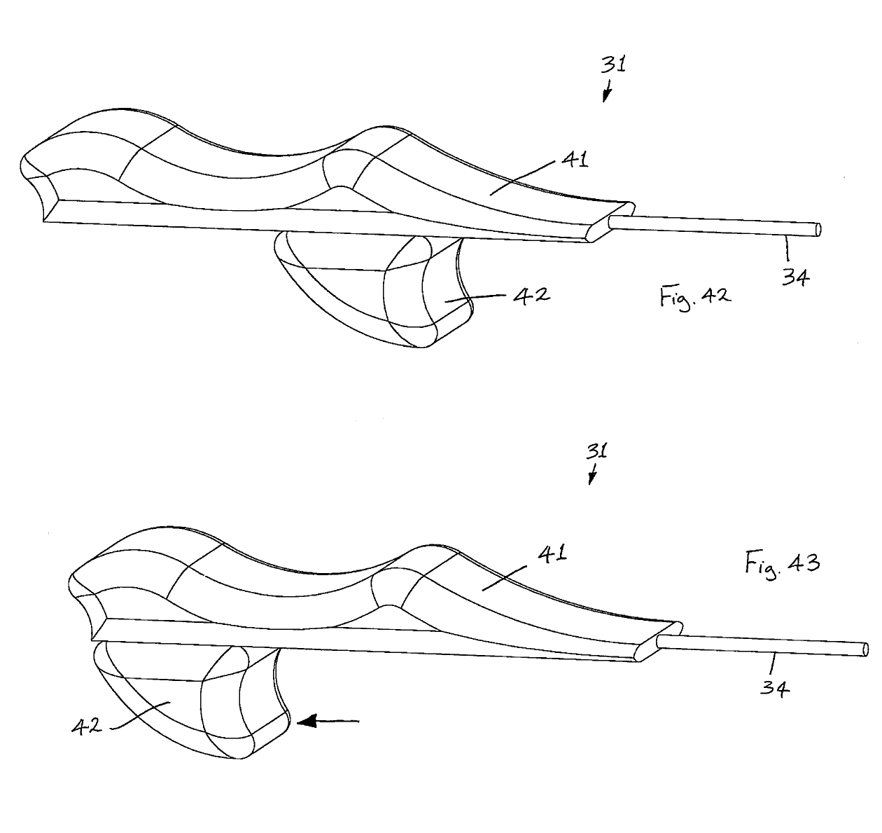

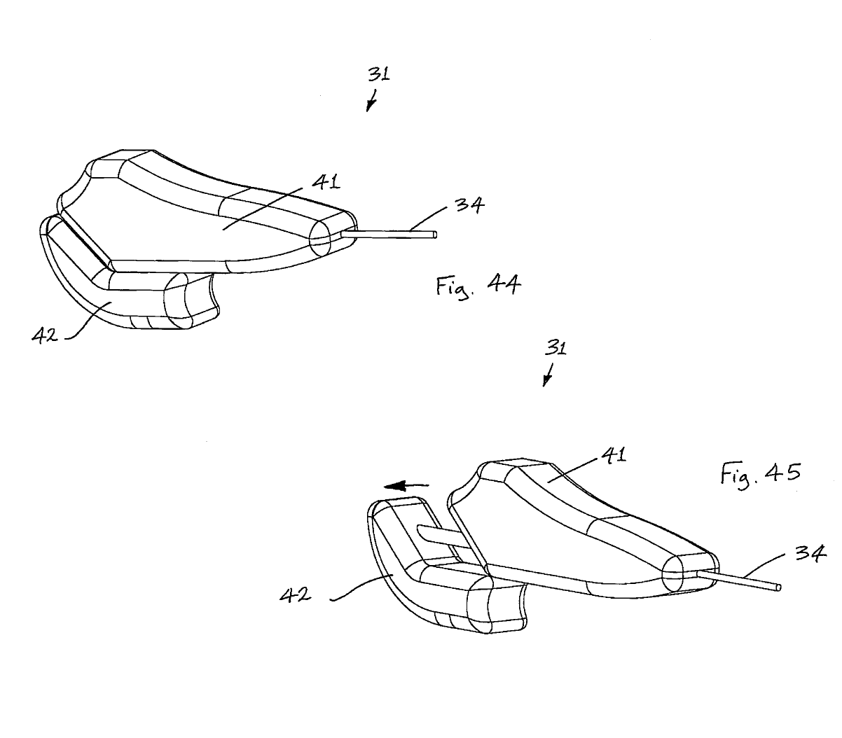

It will be appreciated that the distal handle 41, the proximal handle 42, and the releasable safety clip 43 may be provided in a variety of alternative configurations. For example FIGS. 36 and 37 illustrate a first alternative configuration, FIGS. 38 and 39 illustrate a second alternative configuration, FIGS. 40 and 41 illustrate a third alternative configuration, FIGS. 42 and 43 illustrate a fourth alternative configuration, and FIGS. 44 and 45 illustrate a fifth alternative configuration.

In the embodiments of FIGS. 42 and 43, and FIGS. 44 and 45 no releasable safety clip is provided.

Frictional losses during deployment of an embolic protection filter using the delivery catheter of the invention are low.

The delivery catheter according to the invention is particularly suitable for delivering an embolic protection filter in a downstream direction to a desired location in a vasculature, and deploying the filter at the desired location.

The invention is not limited to the embodiments hereinbefore described, with reference to the accompanying drawings, which may be varied in construction and detail.

1. A delivery catheter, comprising:
a pod, the pod defining a reception space for an embolic protection filter, wherein the pod is constructed of at least one layer of material, the material being at least partially composed of an intractable material.

2. A catheter as claimed in claim 1 wherein the pod is additionally at least partially composed of a non-thermoplastic material.

3. A catheter as claimed in claim 2 wherein the pod is additionally at least partially composed of a thermoset material.

4. A catheter as claimed in claim 3 wherein at least a portion of at least one layer is formed at least partially of polyamide.

5. A catheter as claimed in claim 1 wherein the pod further include a second layer, the second layer formed at least partially of a material having a low coefficient of friction.

6. A catheter as claimed in claim 5 wherein the second layer is at least partially formed of polytetrafluoroethylene.

7. A method of producing a delivery catheter, the method comprising the steps of:
forming a pod at a distal end of a delivery catheter pod, the pod constructed of at least one layer of material, the at least one layer extending from a proximal portion of the pod to a distal portion of the pod; and
flaring at least part of the pod.

8. A method as claimed in claim 7 wherein the pod is flared by inserting a flaring mandrel into the pod.

9. A method as claimed in claim 7 wherein the method comprises the step of heating the pod during flaring.

10. A method as claimed in claim 7 wherein the method comprises the step of necking at least part of the pod.

11. A method as claimed in claim 10 wherein the pod is at least partially necked after flaring.

12. A method as claimed in claim 10 wherein the pod is at least partially necked before flaring.

13. A method as claimed in claim 13 wherein the method comprises the step of disposing emery paper between the pod and the necking clamp before necking.

14. A method as claimed in claim 10 wherein a mandrel is inserted into the pod before necking.

15. A method as claimed in claim 10 wherein the method comprises the step of heating the pod during necking.

16. A method as claimed in claim 10 wherein the method comprises the step of annealing the pod after necking.

17. A method as claimed in claim 7 wherein the method comprises the step of forming the pod from a first layer.

18. A method as claimed in claim 17 wherein the first layer is at least partially formed of an intractable material.

19. A method as claimed in claim 7 wherein the pod is formed of a first layer of material and a second layer of material.

20. A method as claimed in claim 19 wherein the pod is formed by dipping the second layer of material into a quantity of the first layer of material, the first layer of material being in liquid form.
